(19) United States
(12) Patent Application Publication
(10) Pub. No.: US 2007/0016967 A1
(43) Pub. Date: Jan. 18, 2007

(54) Novel IFNgamma-like polypeptides

(75) Inventor: Mark IBBERSON, Gimel (CH)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: Applied Research Systems ARS Holding N.V., Curacao (AN)

(21) Appl. No.: 10/537,555

(22) PCT Filed: Dec. 3, 2003
(86) PCT No.: PCT/EP03/50939

§ 371 Date: Mar. 6, 2006

(30) Foreign Application Priority Data
Dec. 4, 2002 (EP) 02102678.6

(51) Int. Cl. A01K 67/027; C12Q 1/68; C07H 21/04; C12P 21/04; C07K 14/56

(52) U.S. Cl. 800/14; 530/351; 435/6; 435/320.1; 435/325; 536/23.5; 435/69.51

(57) ABSTRACT

The present invention discloses novel open reading frames (ORFs) in human genome encoding for ORFs characterized for polypeptides having at least one activity in human Interferon gamma, and reagents related thereto including variants and fragments of said polypeptides, as well as the encoding nucleic acids and ligands directed against them. The invention provides methods for identifying and preparing these molecules, for manufacturing pharmaceutical compositions containing them, and for using them in the diagnosis, prevention and treatment of diseases.

### FIELD OF THE INVENTION

The present invention relates to nucleic acid sequences identified in human genome as encoding for novel polypeptides, more specifically for novel polypeptides having at least one activity of human interferon gamma.

### BACKGROUND OF THE INVENTION

Interferons (IFNs) are cytokines that play a complex and central role in mammalian immunological response to pathologic events such as infections, immunological disorders, and neoplastic degenerations.

There are two groups of IFNs: type I (IFNalpha and IFNbeta) and type II (IFNgamma, also known as immune interferon). IFNgamma is a cytokine produced by T-lymphocytes and natural killer cells and exists as a homodimer of two noncovalently bound polypeptide subunits, found in different glycosylated forms (Younes H M and Amsden B G, 2002; Boehm U et al., 1997).

IFNgamma is a potent activator of mononuclear phagocytes, capable of affecting immune response by inducing the expression of several molecule, including tumor necrosis factor (TNF), class I/II major histocompatibility complex (MHC) molecules, and the enzymes mediating the respiratory burst which allow macrophages to kill phagocytosed microbes and tumor cells. IFNgamma triggers, by binding its cell surface receptor and activating intracellular signal transduction (JAK-STAT pathway, in particular), not only T and B-lymphocytes differentiation and the cytolytic activity of natural killer (NK) cells, but also the apoptosis or the proliferation of other cell types, such as vascular endothelial cells, also by modulating tryptophan metabolism.

Moreover, polymorphisms in the gene encoding human IFNgamma have been also associated to specific disease states or clinical manifestations that are probably caused by genetically determined aberrant cytokine expression (Vandenbroeck K and Goris A, 2003; WO 02/16631).

The cellular responses to IFNgamma, which can be inhibited and neutralized by the soluble extracellular portion of the IFNgamma receptor (Michiels L et al., 1998) are particularly complex also because this protein coordinates many different cellular events, such apoptosis (Tura B J et al., 2001; Annicchiarico-Petruzzelli M et al., 2001; Pouly S et al., 2000; Luttmann W et al., 2000) or infection (Rottenberg M E et al., 2002; Shtrichman R and Samuel C E, 2001). These activities, which can be cell type-specific or co-regulated with other cytokines such as IL-1beta or TNFalpha, are associated to IFNgamma-induced or IFNgamma-repressed expression of set of genes (Boehm U et al., 1997; Shaw A C et al., 1999).

The properties of IFNgamma have been studied in many disease models. For example, IFNgamma is effective in reducing the formation of extramedullar tumor masses in an animal model of myeloid leukemia (Arai C et al., 1999), in protecting from bacterial sepsis (Zantl N et al., 1998), and to repress virally induced gene expression in combination with TNFalpha (Sethi S K et al., 1997), but it has harmful actions in models for demyelinating disorders (Popko B and Baerwald K D, 1999).

Important therapeutic properties of IFNgamma, alone or in combination with other compounds, have been suggested and/or demonstrated for a broad range of indications including Interstitial Pulmonary Fibrosis (Ziesche R et al., 1999), asthma (WO 01/34180), decay process of bones (EP203580), vascular stenosis (WO 90/03189), Type I diabetes mellitus (WO 95/22328), leukemia (in combination with IFNalpha; U.S. Pat. No. 5,170,591), B cells hyperproliferation-related diseases (in combination with an antibody binding a B-cell antigen; WO 02/102312), steroid resistant condition (U.S. Pat. No. 5,666,312), atopic disorders (WO 91/07984), or septic shock (U.S. Pat. No. 5,198,212; Docke W D et al., 1997). At the same time, compounds antagonizing directly IFNgamma such as soluble receptors or antibodies, or indirectly (at level of its signaling pathway or of its gene expression) such as small molecules, have been described as having therapeutic properties in restenosis (EP1265996) and in controlling autoimmune diseases and hyperimmune response, as in organ rejection (U.S. Pat. No. 6,036,956; EP 1140990; WO 98/28001; WO 94/12531; WO 94/14497; WO 02/98460; WO 99/09055, WO 00/32634).

In cancer immunotherapy, IFNgamma is injected along with irradiated autologous tumor cell, since it acts as an adjuvant and enhances the immune response to the tumor cell challenge. IFNgamma is currently is approved by the Food and Drug Administration (FDA) for limited clinical uses (such as for the reduction of infections associated with chronic granulomatous disease and for delaying progression in patients with malignant osteopetrosis), since this protein also yields significant side effects, such as fever, fatigue, nausea, and neurotoxicity.

These limitations, probably due to the expression of IFNgamma receptors on the surface of almost all types of human cells and the consequent excessive signaling activities (Bach E A et al., 1997), have prompted the development of alternative forms and delivering systems for this cytokine to achieve more acceptable results. Various naturally-occurring or synthetic forms of the human IFNgamma have been described, having longer or shorter N-/C-terminal sequences, or mutated in specific residues for improving specific properties such as heat-stability (WO 97/11179) or glycosylation (WO 01/36001; WO 02/81507). Peptides derived from human IFNgamma having properties similar to the complete sequence have been also disclosed (U.S. Pat. No. 6,120,762).

The literature provides many examples of different approaches for characterizing novel proteins by making use of bioinformatics analysis of transcripts. For example, GB patent application No. 0130720.6 (published as WO 03/055913) discloses a polypeptide sequence, called INSP037, matching structural features of IFNgamma.

Since the actual content in DNA sequence in human genome encoding for IFNs (and for any other protein family) is still unknown, the possibility still exists to identify DNA sequence encoding polypeptide having IFNgamma-like structure and activity by applying alternative homology/structural criteria to the totality of Open Reading Frames (ORFs, that is, genomic sequences containing consecutive triplets of nucleotides coding for amino acids, not interrupted by a termination codon and potentially translatable in a polypeptide) present in human genome.

### SUMMARY OF THE INVENTION

The invention is based upon the identification of Open Reading Frames (ORFs) in human genome encoding novel IFNgamma-like polypeptides on the basis of the homology with INSP037, but that can be grouped under a novel consensus sequence called pIFNFHcon.

In particular, the invention provides pIFNFH polypeptides having the amino acid sequence given by SEQ ID NO: 2, 4, 6, 8, 10, 12, 14, 16, 18, 20, 22, 24, 26, 28, 30, 32, 34, 36, 38, and 40, as novel polypeptides having at least one activity of human IFNgamma. The invention includes also the nucleic acids encoding them, vectors containing such nucleic acids, and cell containing these vectors or nucleic acids, as well as other related reagents such as fusion proteins and ligands, which may act as antagonists.

The invention provides methods for identifying and making these molecules, for preparing pharmaceutical compositions containing them, and for their use in the diagnosis, prevention and treatment of diseases where compounds having at least one activity of human IFNgamma, or their antagonists, may provide positive effects.

### DESCRIPTION OF THE FIGURES

FIG. 1: alignment of IFNFH01 ORF (SEQ ID NO: 1) with pIFNFH01 protein sequence (SEQ ID NO: 2). The residues found identical in INSP037 are underlined (71% of identity with INSP037). The arrows indicate the position of the primers CL_IFNFH01_{—}5 (forward. SEQ ID NO: 41) and CL_IFNFH01_{—}3 (reverse; SEQ ID NO: 42) in the ORF sequence.

FIG. 2: alignment of IFNFH03 ORF (SEQ ID NO: 3) with pIFNFH03 protein sequence (SEQ ID NO: 4). The residues found identical in INSP037 are underlined (73.5% of identity with INSP037). The arrows indicate the position of the primers CL_IFNFH03_{—}5 (forward; SEQ ID NO: 43) and CL_IFNFH03_{—}3 (reverse; SEQ ID NO: 44) in the ORF sequence.

FIG. 3: alignment of IFNFH04 ORF (SEQ ID NO: 5) with pIFNFH04 protein sequence (SEQ ID NO: 6). The residues found identical in INSP037 are underlined (73.5% of identity with INSP037). The arrows indicate the position of the primers CL_IFNFH04_{—}5 (forward; SEQ ID NO: 45) and CL_IFNFH04_{—}3 (reverse; SEQ ID NO: 46) in the ORF sequence.

FIG. 4: alignment of IFNFH08 ORF (SEQ ID NO: 7) with pIFNFH08 protein sequence (SEQ ID NO: 8). The residues found identical in INSP037 are underlined (78.5% of identity with INSP037). The arrows indicate the position of the primers CL_IFNFH08_{—}5 (forward; SEQ ID NO: 47) and CL_IFNFH08_{—}3 (reverse; SEQ ID NO: 48) in the ORF sequence.

FIG. 5: alignment of IFNFH10 ORF (SEQ ID NO: 9) with pIFNFH10 protein sequence (SEQ ID NO: 10). The residues found identical in INSP037 are underlined (69.5% of identity with INSP037). The arrows indicate the position of the primers CL_IFNFH10_{—}5 (forward; SEQ ID NO: 49) and CL_IFNFH10_{—}3 (reverse; SEQ ID NO: 50) in the ORF sequence.

FIG. 6: alignment of IFNFH11 ORF (SEQ ID NO: 11) with pIFNFH11 protein sequence (SEQ ID NO: 12). The residues found identical in INSP037 are underlined (73.5% of identity with INSP037). The arrows indicate the position of the primers CL_IFNFH11_{—}5 (forward; SEQ ID NO: 51) and CL_IFNFH11_{—}3 (reverse; SEQ ID NO: 52) in the ORF sequence.

FIG. 7: alignment of IFNFH12 ORF (SEQ ID NO: 13) with pIFNFH12 protein sequence (SEQ ID NO: 14). The residues found identical in INSP037 are underlined (73.5% of identity with INSP037). The arrows indicate the position of the primers CL_IFNFH12_{—}5 (forward; SEQ ID NO: 53) and CL_IFNFH12_{—}3 (reverse; SEQ ID NO: 54) in the ORF sequence.

FIG. 8: alignment of IFNFH13 ORF (SEQ ID NO: 15) with pIFNFH13 protein sequence (SEQ ID NO: 16). The residues found identical in INSP037 are underlined (69.5% of identity with INSP037). The arrows indicate the position of the primers CL_IFNFH13_{—}5 (forward; SEQ ID NO: 55) and CL_IFNFH13_{—}3 (reverse; SEQ ID NO: 56) in the ORF sequence.

FIG. 9: alignment of IFNFH14 ORF (SEQ ID NO: 17) with pIFNFH14 protein sequence (SEQ ID NO: 18). The residues found identical in INSP037 are underlined (71% of identity with INSP037). The arrows indicate the position of the primers CL_IFNFH14_{—}5 (forward; SEQ ID NO: 57) and CL_IFNFH14_{—}3 (reverse; SEQ ID NO: 58) in the ORF sequence.

FIG. 10: alignment of IFNFH15 ORF (SEQ ID NO: 19) with pIFNFH15 protein sequence (SEQ ID NO: 20). The residues found identical in INSP037 are underlined (71% of identity with INSP037). The arrows indicate the position of the primers CL_IFNFH15_{—}5 (forward; SEQ ID NO: 59) and CL_IFNFH15_{—}3 (reverse; SEQ ID NO: 60) in the ORF sequence.

FIG. 11: alignment of IFNFH20 ORF (SEQ ID NO: 21) with pIFNFH20 protein sequence (SEQ ID NO: 22). The residues found identical in INSP037 are underlined (67% of identity with INSP037). The arrows indicate the position of the primers CL_IFNFH20_{—}5 (forward; SEQ ID NO: 61) and CL_IFNFH20_{—}3 (reverse; SEQ ID NO: 62) in the ORF sequence.

FIG. 12: alignment of IFNFH23 ORF (SEQ ID NO: 23) with pIFNFH23 protein sequence (SEQ ID NO: 24). The residues found identical in INSP037 are underlined (72% of identity with INSP037). The arrows indicate the position of the primers CL_IFNFH23_{—}5 (forward; SEQ ID NO: 63) and CL_IFNFH23_{—}3 (reverse; SEQ ID NO: 64) in the ORF sequence.

FIG. 13: alignment of IFNFH25 ORF (SEQ ID NO: 25) with pIFNFH25 protein sequence (SEQ ID NO: 26). The residues found identical in INSP037 are underlined (70% of identity with INSP037).

FIG. 14: alignment of IFNFH27 ORF (SEQ ID NO: 27) with pIFNFH27 protein sequence (SEQ ID NO: 28). The residues found identical in INSP037 are underlined (68% of identity with INSP037). The arrows indicate the position of the primers CL_IFNFH27_{—}5 (forward; SEQ ID NO: 65) and CL_IFNFH27_{—}3 (reverse; SEQ ID NO: 66) in the ORF sequence.

FIG. 15: alignment of IFNFH31 ORF (SEQ ID NO: 29) with pIFNFH31 protein sequence (SEQ ID NO: 30). The residues found identical in INSP037 are underlined (68% of identity with INSP037). The arrows indicate the position of the primers CL_IFNFH31_{—}5 (forward; SEQ ID NO: 67) and CL_IFNFH31_{—}3 (reverse; SEQ ID NO: 68) in the ORF sequence.

FIG. 16: alignment of IFNFH32 ORF (SEQ ID NO: 31) with pIFNFH32 protein sequence (SEQ ID NO: 32). The residues found identical in INSP037 are underlined (70% of identity with INSP037). The arrows indicate the position of the primers CL_IFNFH32_{—}5 (forward; SEQ ID NO: 69) and CL_IFNFH32_{—}3 (reverse; SEQ ID NO: 70) in the ORF sequence.

FIG. 17: alignment of IFNFH36 ORF (SEQ ID NO: 33) with pIFNFH36 protein sequence (SEQ ID NO: 34). The residues found identical in INSP037 are underlined (72% of identity with INSP037). The arrows indicate the position of the primers CL_IFNFH36_{—}5 (forward; SEQ ID NO: 71) and CL_IFNFH36_{—}3 (reverse; SEQ ID NO: 72) in the ORF sequence.

FIG. 18: alignment of IFNFH37 ORF (SEQ ID NO: 35) with pIFNFH37 protein sequence (SEQ ID NO: 36). The residues found identical in INSP037 are underlined (76% of identity with INSP037). The arrows indicate the position of the primers CL_IFNFH37_{—}5 (forward; SEQ ID NO: 73) and CL_IFNFH37_{—}3 (reverse; SEQ ID NO: 74) in the ORF sequence.

FIG. 19: alignment of IFNFH39 ORF (SEQ ID NO: 37) with pIFNFH39 protein sequence (SEQ ID NO: 38). The residues found identical in INSP037 are underlined (70% of identity with INSP037). The arrows indicate the position of the primers CL_IFNFH39_{—}5 (forward; SEQ ID NO: 75) and CL_IFNFH39_{—}3 (reverse; SEQ ID NO: 76) in the ORF sequence.

FIG. 20: alignment of IFNFH42 ORF (SEQ ID NO: 39) with pIFNFH42 protein sequence (SEQ ID NO: 40). The residues found identical in INSP037 are underlined (67% of identity with INSP037). The arrows indicate the position of the primers CL_IFNFH42_{—}5 (forward; SEQ ID NO: 77) and CL_IFNFH42_{—}3 (reverse; SEQ ID NO: 78) in the ORF sequence.

FIG. 21: alignment of the human IFN gamma-like INSP037 (SEQ ID NO: 155) with the protein sequences of the invention, including pIFNFHs and the consensus sequence pIFNFHcon (SEQ ID NO:156), which is identified as the region common to INSP037 and pIFNFHs I(boxed area). The residues characterizing pIFNFHcon from INSP037 are indicated in pIFNFHcon sequence in bold (Ala10, Gly12, Arg26, Ala31, Lys35, Phe47, Gln55, Glu57, Lys63, Ile75; numbering bullets are located each 10 amino acids). The residues in INSP037 and in the pIFNFHs sequences that are not conserved in pIFNFHcon are underlined.

FIG. 22: alignment of pIFNFHcon and INSP037 with the most similar sequences known from the prior art, that are named according Derwent DGENE database indexing as ABG00143 (SEQ ID NO: 157) and AAM70428 (SEQ ID NO: 158). The residues characterizing pIFNFHcon from INSP037 are indicated in pIFNFHcon in bold.

FIG. 23: map of the expression vector pEAK12D.

### DETAILED DESCRIPTION OF THE INVENTION

A sequence analysis of human genome to identify homologs of INSP037, a polypeptide sequence matching structural features of IFNgamma (WO 03/055913) allowed to identify a series of polypeptides that, even if they are similar to INSP037, have common sequence features allowing to group them under a novel consensus sequence of 75 amino acids, called pIFNFHcon, characterizing sequences predicted to have at least one activity of human IFNgamma

The main object of the present invention are isolated polypeptides presenting at least one activity of human IFNgamma, and comprising a sequence having:
a) at least 80% of homology with the complete sequence of pIFNFHcon (SEQ ID NO: 156); and
b) no more than nine non-conservative mutations in the positions corresponding to Ala10, Gly12, Arg26, Ala31, Lys35, Phe47, Gln55, Glu57, Lys63, and Ile75 in pIFNFHcon.

The totality of amino acid sequences obtained by translating the known ORFs in the human genome were challenged using INSP037 protein sequence, and the positive hits were further selected on the basis of sequence length and amino acid conservation comparable to INSP037 and/or human IFNgamma. Therefore, the novel polypeptides of the invention can be predicted to have at least one of the biological activities of human IFNgamma.

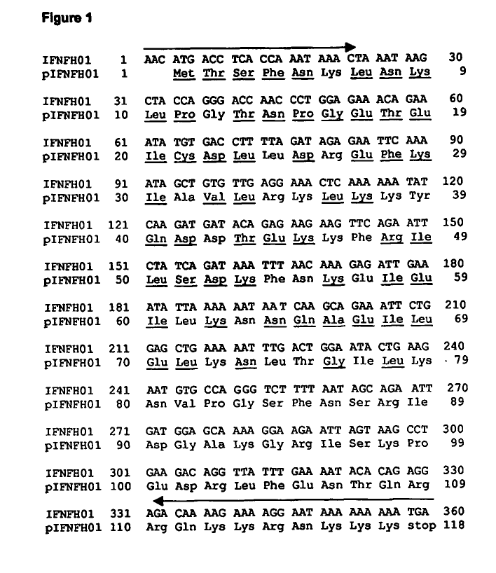

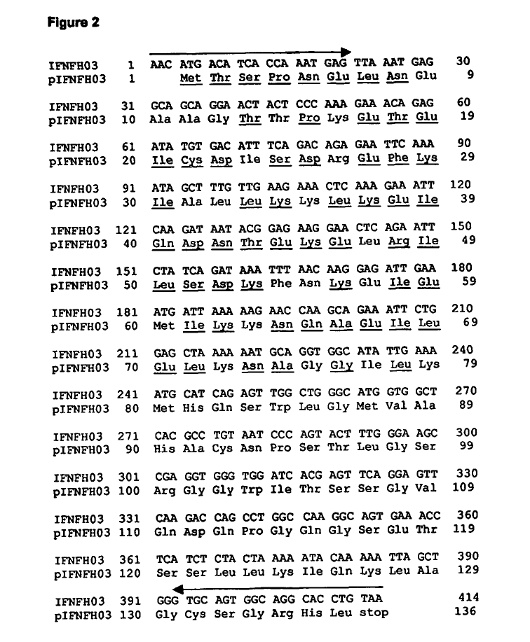

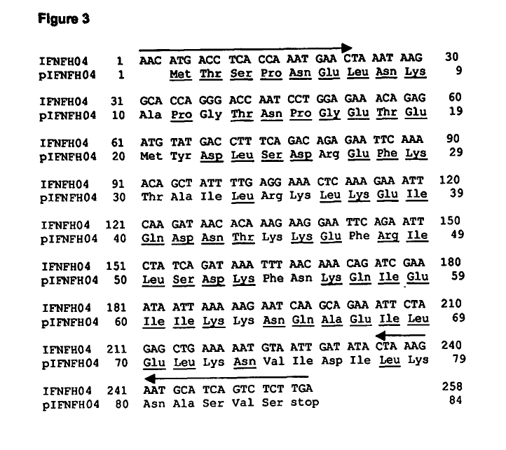

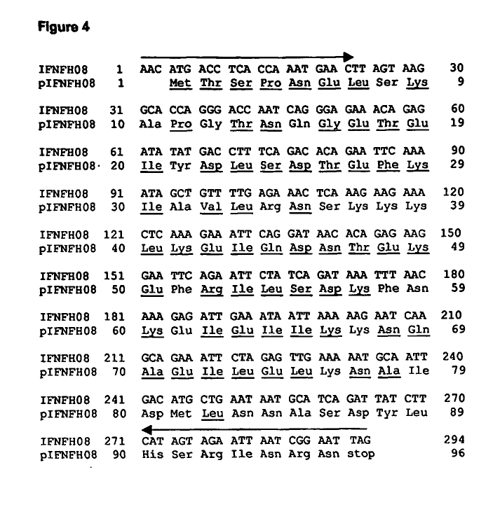

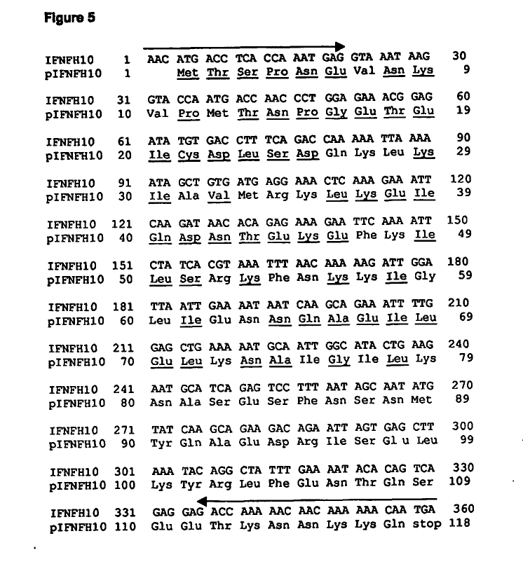

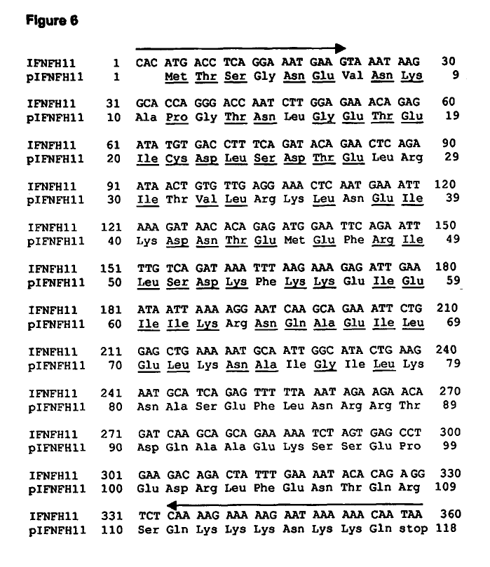

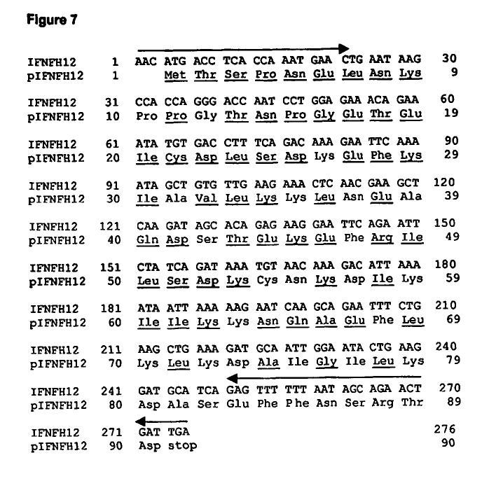

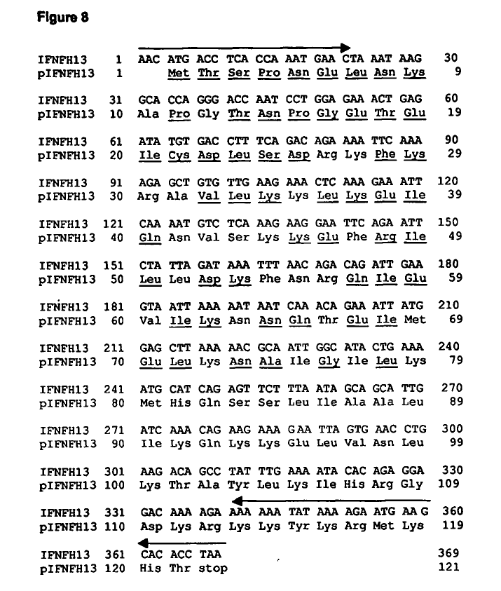

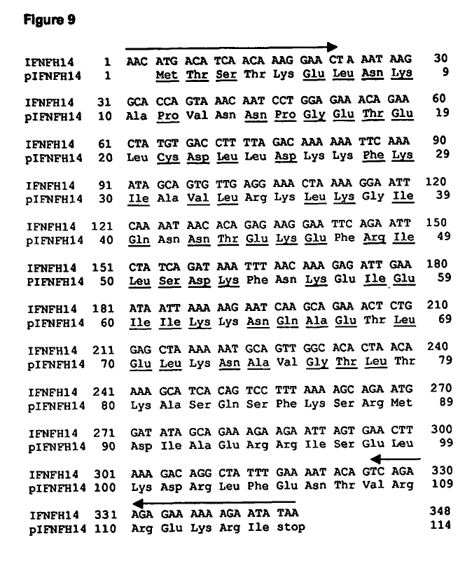

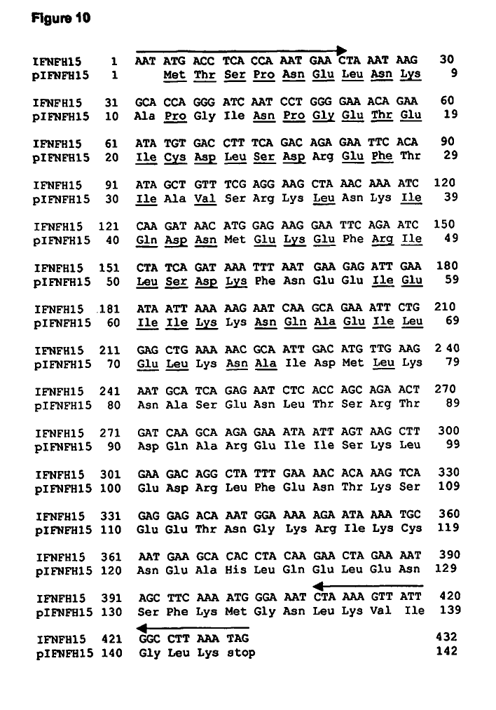

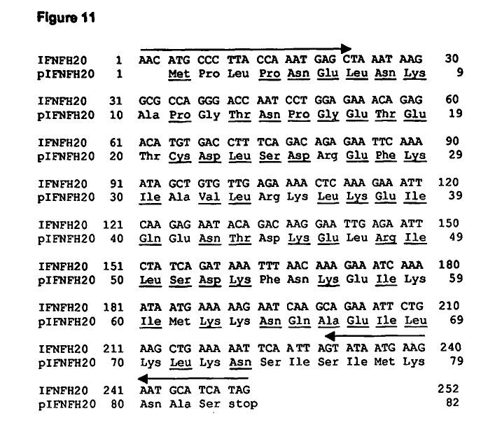

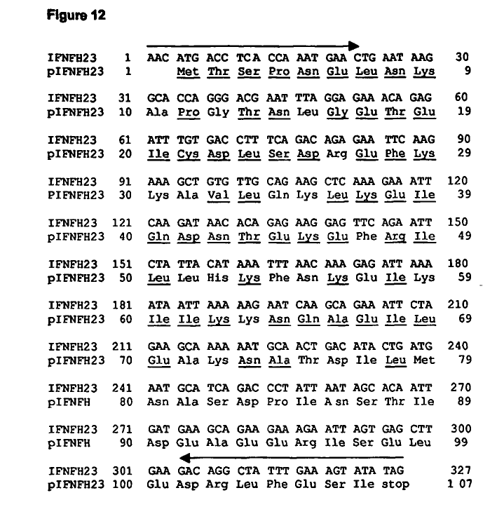

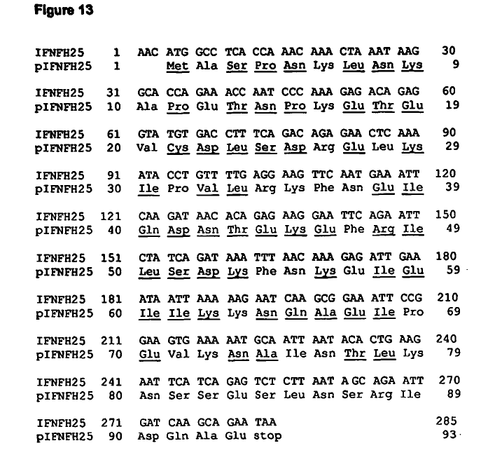

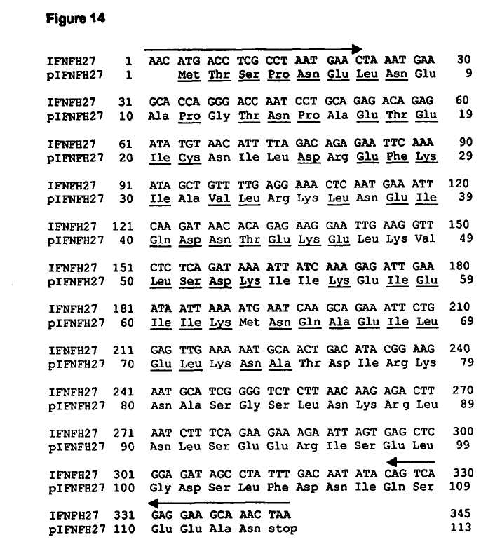

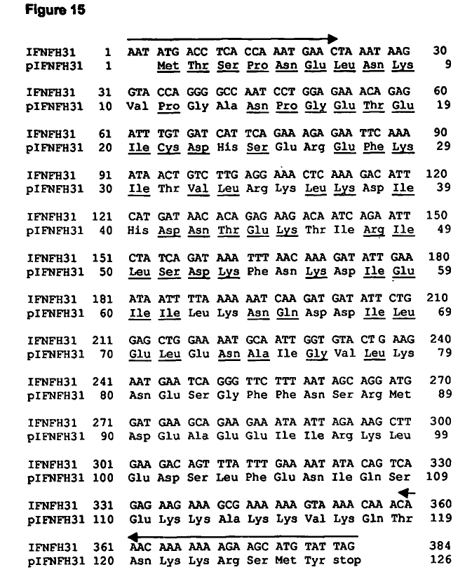

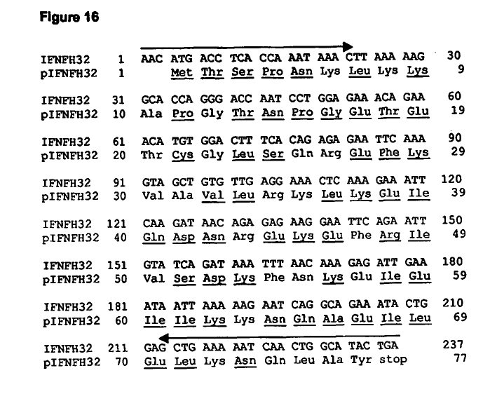

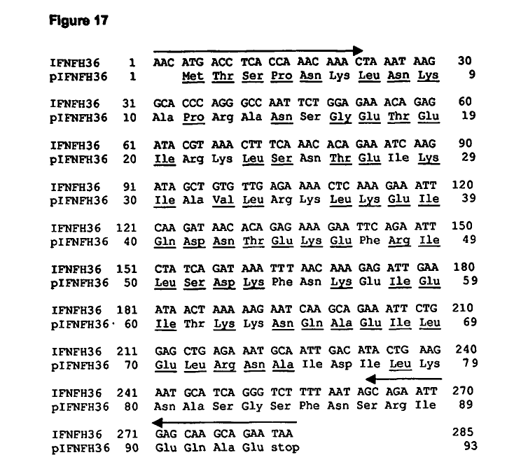

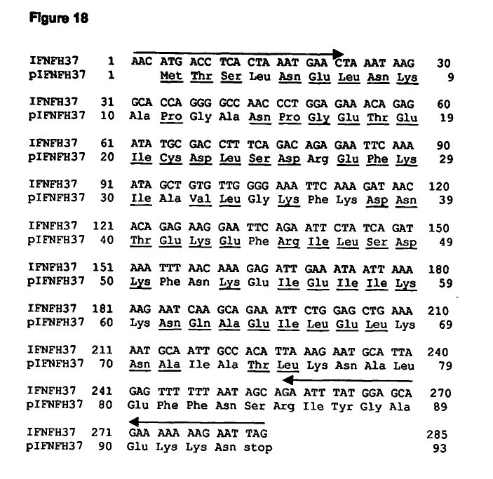

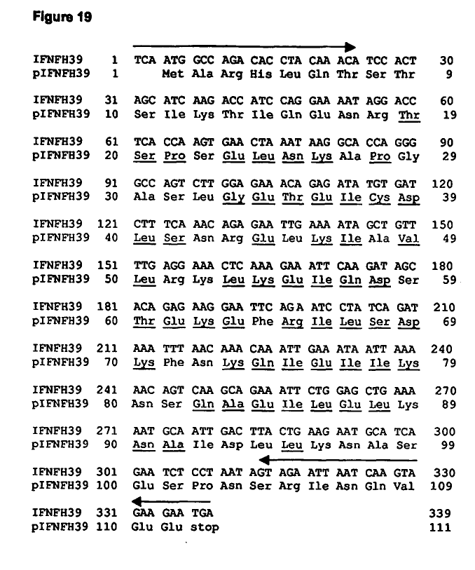

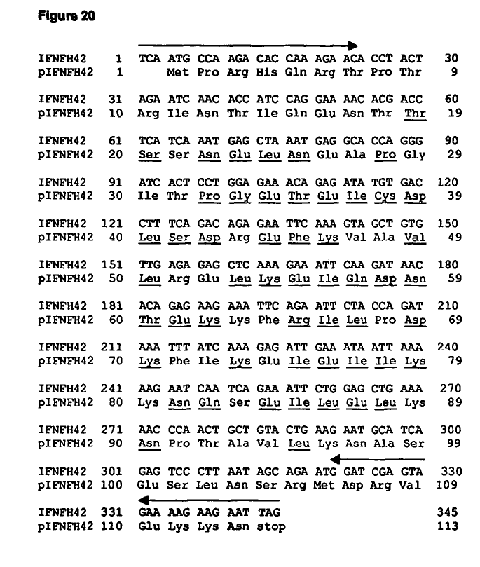

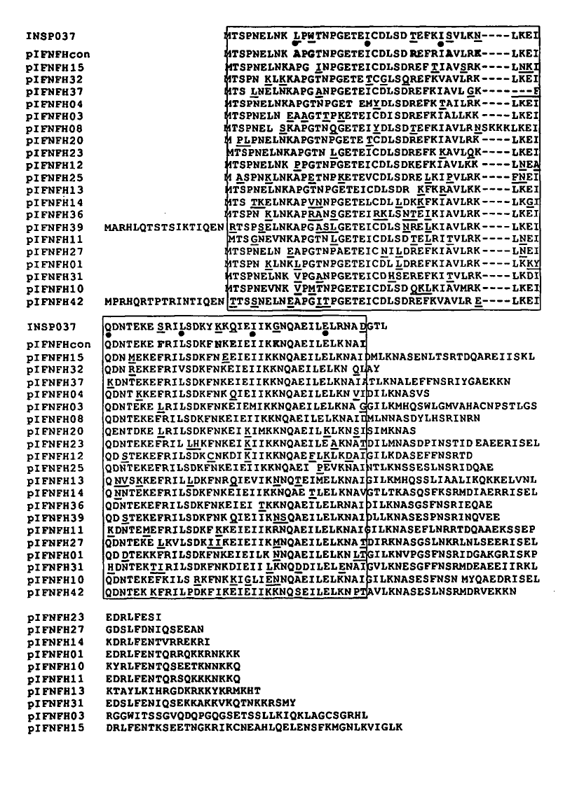

The novel polypeptides pIFNFH01 (SEQ ID NO: 2; FIG. 1), pIFNFH03 (SEQ ID NO: 4 FIG. 2), pIFNFH04 (SEQ ID NO: 6; FIG. 3), pIFNFH08 (SEQ ID NO: 8; FIG. 4), pIFNFH10 (SEQ ID NO: 10; FIG. 5), pIFNFH11 (SEQ ID NO: 12; FIG. 6), pIFNFH12 (SEQ ID NO: 14; FIG. 7), pIFNFH13 (SEQ ID NO: 16; FIG. 8), pIFNFH14 (SEQ ID NO: 18; FIG. 9), pIFNFH15 (SEQ ID NO: 20; FIG. 10), pIFNFH20 (SEQ ID NO: 22; FIG. 11), pIFNFH23 (SEQ ID NO: 24; FIG. 12), pIFNFH25 (SEQ ID NO: 26; FIG. 13), pIFNFH27 (SEQ ID NO: 28; FIG. 14), pIFNFH31 (SEQ ID NO: 30; FIG. 15), pIFNFH32 (SEQ ID NO: 32; FIG. 16), pIFNFH36 (SEQ ID NO: 34; FIG. 17), pIFNFH37 (SEQ ID NO: 36; FIG. 18), pIFNFH39 (SEQ ID NO: 38; FIG. 19), and pIFNFH42 (SEQ ID NO: 40; FIG. 20) were identified on the basis of the comparable length and the sequence homology with INSP037, but further distinctions can be made amongst pIFNFHs on the basis of the consensus sequence pIFNFHcon (FIG. 21).

A first group of pIFNFHs includes polypeptides that comprise a sequence having at least 80% of homology with the complete sequence of pIFNFHcon and no non-conservative mutations in the positions corresponding to Ala10, Gly12, Arg26, Ala31, Lys35, Phe47, Gln55, Glu57, Lys63, and Ile75 in pIFNFHcon. Examples of such sequences are pIFNFH15 (SEQ ID NO: 20), pIFNFH32 (SEQ ID NO: 32), and pIFNFH37 (SEQ ID NO: 36).

A second group of pIFNFHs includes polypeptides that comprise a sequence having at least 80% of homology with the complete sequence of pIFNFHcon and one or two non-conservative mutations in the positions corresponding to Ala10, Gly12, Arg26, Ala31, Lys35, Phe47, Gln55, Glu57, Lys63, and Ile75 in pIFNFHcon. Examples of such sequences are pIFNFH04 (SEQ ID NO: 6), pIFNFH03 (SEQ ID NO: 4), pIFNFH08 (SEQ ID NO: 8), pIFNFH20 (SEQ ID NO: 22), pIFNFH23 (SEQ ID NO: 24), pIFNFH12 (SEQ ID NO: 14), pIFNFH25 (SEQ ID NO: 26), pIFNFH13 (SEQ ID NO: 16), pIFNFH14 (SEQ ID NO: 18), pIFNFH36 (SEQ ID NO: 34), and pIFNFH39 (SEQ ID NO: 38).

A third group of pIFNFHs includes polypeptides that comprise a sequence having at least 80% of homology with the complete sequence of pIFNFHcon and three, four, or five non-conservative mutations in the positions corresponding to Ala10, Gly12, Arg26, Ala31, Lys35, Phe47, Gln55, Glu57, Lys63, and Ile75 in pIFNFHcon. Examples of such sequences are pIFNFH11 (SEQ ID NO: 12), pIFNFH27 (SEQ ID NO: 28), pIFNFH01 (SEQ ID NO: 2), pIFNFH31 (SEQ ID NO: 30), pIFNFH10 (SEQ ID NO: 10), and pIFNFH42 (SEQ ID NO: 40).

Sequences homologous to pIFNFHcon, and to pIFNFHs in general, can be identified and/or designed using commonly available bioinformatic tools (Mulder N J and Apweiler R, 2002; Rehm B H, 2001), by measuring the percentage over the segment of 75 amino acids corresponding to the region conserved in p IFNFHs, and characterized in the present invention as pIFNFHcon (FIG. 21).

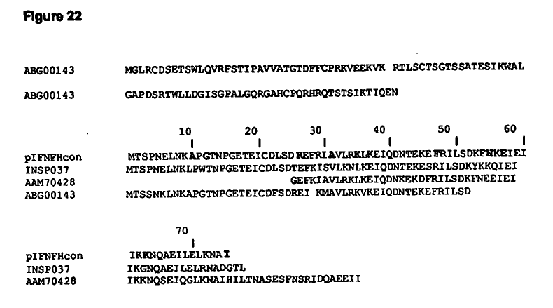

The consensus sequence pIFNFHcon, in connection with the identification of specific residues to be conserved, characterizes pIFNFHs and allows to make a clear distinction not only between pIFNFHs and INSP037 but also between pIFNFHs and sequences disclosed in the literature that are homologous to a portion of INSP037 and of pIFNFHs, and identified as ABG00143 (SEQ ID NO: 157; WO 01/75067) and AAM70428 (SEQ ID NO: 158; WO 01/57276) in FIG. 22. In accordance with the present invention, a “non-conservative mutation” is any change in the sequence not involving or a “conservative” or “safe” substitution. A “conservative” mutation introduces an amino acids having sufficiently similar chemical properties (eg a basic, positively charged amino acid should be replaced by another basic, positively charged amino acid), in order to preserve the structure and the biological function of the molecule. Therefore, the phrase “non-conservative mutation” encompasses also deletions and insertions. The groups of synonymous amino acids that can be used for determining sequence homology and conservative mutations are shown in Table I.

Specific non-conservative mutations may be introduced in the polypeptides of the invention with different purposes, for example, the elimination of immunogenic epitopes, the alteration of binding properties, the alteration of the glycosylation pattern, or the improvement of protein stability (van den Burg B and Eijsink V, 2002; Robinson C R, 2002: WO 02/05146; WO 00/34317; WO 98/52976).

In addition to such sequences, a series of polypeptides forms part of the disclosure of the invention, such as variants, mature forms, or active fragments of the amino acid sequences SEQ ID NO: 2, 4, 6, 8, 10, 12, 14, 16, 18, 20, 22, 24, 26, 28, 30, 32, 34, 36, 38, and 40.

The variants may correspond to naturally occurring allelic variants of the sequences SEQ ID NO: 2, 4, 6, 8, 10, 12, 14, 16, 18, 20, 22, 24, 26, 28, 30, 32, 34, 36, 38, and 40, as the ones possibly resulting from the translation of one or more single nucleotide polymorphisms.

Mature forms and active fragments of the amino acid sequences SEQ ID NO: 2, 4, 6, 8, 10, 12, 14, 16, 18, 20, 22, 24, 26, 28, 30, 32, 34, 36, 38, and 40, should have at least one of the biological activities of human IFNgamma, as reviewed (Bach E A et al., 1997; Boehm U et al., 1997), or shown in the in the literature cited in the Background of the Invention. These activities can be detected either at the level of physiologic or cellular events (such as immune/antiviral response, antigen presentation, respiratory burst, leukocyte-endothelial interactions, or cell proliferation/apoptosis), as well as at the level of induction or repression of the expression of specific genes, or set of genes.

Mature forms and active fragments can result from natural or artificial post-transcriptional or post-translational events. For example, truncated proteins can be generated by genetic engineering and expressed in host cells, or by a proteolytic processing leading to the removal of N-terminal sequences (by signal peptidases and other proteolytic enzymes). Other alternative mature forms can also result from the addition of chemical groups such as sugars or phosphates.

Fragments should present deletions of terminal or internal amino acids not altering their function, and should involve generally a few amino acids, e.g., under ten, and preferably under three, without removing or displacing amino acids which are critical to the conformation of the active protein. In particular the ones conserved in pIFNFHs and indicated in the consensus sequence pIFNFHcon. Alternatively, the fragments may correspond to a specific portion of the sequence as shown for IFNgamma-related peptides disclosed in the literature (U.S. Pat. No. 6,120,762).

All the above indicated variants can be natural, being identified in organisms other than humans, or artificial, being prepared by chemical synthesis, by site-directed mutagenesis techniques, or any other known technique suitable thereof, which provide a finite set of substantially corresponding mutated or shortened peptides or polypeptides which can be routinely obtained and tested by one of ordinary skill in the art using the teachings presented in the prior art.

The present patent application discloses also fusion proteins comprising any of the polypeptides described above. These polypeptides should contain at least protein sequence heterologous to the one disclosed in the present patent application, without significatively impairing the IFNgamma-related activity and possibly providing additional properties. Examples of such properties are an easier purification procedure, a longer lasting half-life in body fluids, an additional binding moiety, the maturation by means of an endoproteolytic digestion, or extracellular localization. This latter feature is of particular importance for defining a specific group of fusion or chimeric proteins included in the above definition since it allows the claimed molecules to be localized in the space where not only isolation and purification of these polypeptides is facilitated, but also where generally IFNgamma and its receptors interact.

Design of the moieties, ligands, and linkers, as well methods and strategies for the construction, purification, detection and use of fusion proteins are disclosed in the literature (Nilsson J et al., 1997; Methods Enzymol, Vol. 326-328, Academic Press, 2000). The preferred one or more protein sequences which can be comprised in the fusion proteins belong to these protein sequences: membrane-bound protein, immunoglobulin constant region, multimerization domains, extracellular proteins, signal peptide-containing proteins, export signal-containing proteins. Features of these sequences and their specific uses are disclosed in a detailed manner, for example, for albumin fusion proteins (WO 01/77137), fusion proteins including multimerization domain (WO 01/02440, WO 00/24782), immunoconjugates (Garnett M C, 2001), or fusion protein providing additional sequences which can be used for purifying the recombinant products by affinity chromatography (Constans A, 2002; Burgess R R and Thompson N E, 2002; Lowe C R et al., 2001; Sheibani N, 1999).

The novel amino acid sequences disclosed in the present patent application can be used to provide different kind of reagents and molecules, in particular ligands binding specifically to them. These molecules can be natural or artificial, very different from the chemical point of view (binding proteins, antibodies, molecularly imprinted polymers), and can be produced by applying the teachings in the art (WO 02/74938; Kuroiwa Y et al., 2002; Haupt K, 2002; van Dijk M A and van de Winkel J G, 2001; Gavilondo J V and Larrick J W, 2000).

Examples of these compounds are binding proteins or antibodies that can be identified using their full sequence or specific fragments, such as antigenic determinants. Peptide libraries can be also used for screening and characterizing antibodies or other proteins (Tribbick G, 2002) that bind the claimed amino acid sequences, and for identifying alternative forms of the polypeptides of the invention having similar properties.

As shown for IFNgamma in the literature cited in the Background of the Invention, such ligands can antagonize or inhibit the IFNgamma-related activity of the polypeptide of the invention, providing molecules having several potential applications related to the neutralization of one or more pIFNFH polypeptides.

Common and efficient ligands are represented by antibodies, which can be in the form of a monoclonal, polygonal, or humanized antibody, or of an antigen-binding fragment. Alternatively, the ligand can be a membrane-bound receptor having signaling properties, as shown for IFNgamma receptor (Bach E A et al., 1997; Michiels L et al., 1998), and in particular of extracellular domain of a membrane-bound protein that can be found in the circulation as a soluble receptor, or generated synthetically.

The polypeptides of the present invention can be provided also in the form of active fractions, precursors, salts, or derivatives

The term “fraction” refers to any fragment of the polypeptidic chain of the compound itself, alone or in combination with related molecules or residues bound to it, for example residues of sugars or phosphates, or aggregates of the original polypeptide or peptide. Such molecules can result also from other modifications which do not normally alter primary sequence, for example in vivo or in vitro chemical derivativization of peptides (acetylation or carboxylation), those made by modifying the pattern of phosphorylation (introduction of phosphotyrosine, phosphoserine, or phosphothreonine residues) or glycosylation (by exposing the peptide to enzymes which affect glycosylation e.g., mammalian glycosylating or deglycosylating enzymes) of a peptide during its synthesis and processing or in further processing steps.

The “precursors” are compounds which can be converted into the compounds of present invention by metabolic and enzymatic processing prior or after the administration to the cells or to the body.

The term “salts” herein refers to both salts of carboxyl groups and to acid addition salts of amino groups of the polypeptides of the present invention. Salts of a carboxyl group may be formed by means known in the art and include inorganic salts, for example, sodium, calcium, ammonium, ferric or zinc salts, and the like, and salts with organic bases as those formed, for example, with amines, such as triethanolamine, arginine or lysine, piperidine, procaine and the like. Acid addition salts include, for example, salts with mineral acids such as, for example, hydrochloric acid or sulfuric acid, and salts with organic acids such as, for example, acetic acid or oxalic acid. Any of such salts should have substantially similar activity to the peptides and polypeptides of the invention or their analogs.

The term “derivatives” as herein used refers to derivatives which can be prepared from the functional groups present on the lateral chains of the amino acid moieties or on the amino- or carboxy-terminal groups according to known methods. Such molecules can result also from other modifications which do not normally alter primary sequence, for example in vivo or in vitro chemical derivativization of polypeptides (acetylation or carboxylation), those made by modifying the pattern of phosphorylation (introduction of phosphotyrosine, phosphoserine, or phosphothreonine residues) or glycosylation (by exposing the polypeptide to mammalian glycosylating enzymes) of a peptide during its synthesis and processing or in further processing steps. Alternatively, derivatives may include esters or aliphatic amides of the carboxyl-groups and N-acyl derivatives of free amino groups or O-acyl derivatives of free hydroxyl-groups and are formed with acyl-groups as for example alcanoyl- or aryl-groups.

The generation of the derivatives may involve a site-directed modification of an appropriate residue, in an internal or terminal position. The residues used for attachment should they have a side-chain amenable for polymer attachment (i.e., the side chain of an amino acid bearing a functional group, e.g., lysine, aspartic acid, glutamic acid, cysteine, histidine, etc.). Alternatively, a residue having a side chain amenable for polymer attachment can replace an amino acid of the polypeptide, or can be added in an internal or terminal position of the polypeptide. Also, the side chains of the genetically encoded amino acids can be chemically modified for polymer attachment, or unnatural amino acids with appropriate side chain functional groups can be employed. The preferred method of attachment employs a combination of peptide synthesis and chemical ligation. Advantageously, the attachment of a water-soluble polymer will be through a biodegradable linker, especially at the amino-terminal region of a protein. Such modification acts to provide the protein in a precursor (or “pro-drug”) form, that, upon degradation of the linker releases the protein without polymer modification.

Polymer attachment may be not only to the side chain of the amino acid naturally occurring in a specific position of the antagonist or to the side chain of a natural or unnatural amino acid that replaces the amino acid naturally occurring in a specific position of the antagonist, but also to a carbo hydrate or other moiety that is attached to the side chain of the amino acid at the target position. Rare or unnatural amino acids can be also introduced by expressing the protein in specifically engineered bacterial strains (Bock A, 2001).

The term “active” means that such alternative compounds should maintain the functional features of the polypeptides of the present invention, and should be as well useful for pharmacological or any other type of application.

The polypeptides and the polypeptide-based derived reagents described above can be also in other alternative forms, according to the desired method of use and/or production, such as active conjugates or complexes with a molecule chosen amongst radioactive labels, fluorescent labels, biotin, or cytotoxic agents.

Specific molecules, such as peptide mimetics, can be also designed on the sequence and/or the structure of a polypeptide of the invention defined by the consensus sequence pIFNFHcon. Peptide mimetics (also called peptidomimetics) are peptides chemically modified at the level of amino acid side chains, of amino acid chirality, and/or of the peptide backbone. These alterations are intended to provide agonists or antagonists of the polypeptides of the invention with improved preparation, potency and/or pharmacokinetics features.

For example, when the peptide is susceptible to cleavage by peptidases following injection into the subject is a problem, replacement of a particularly sensitive peptide bond with a non-cleavable peptide mimetic can provide a peptide more stable and thus more useful as a therapeutic compound. Similarly, the replacement of an L-amino acid residue is a standard way of rendering the peptide less sensitive to proteolysis, and finally more similar to organic compounds other than peptides. Also useful are amino-terminal blocking groups such as t-butyloxycarbonyl, acetyl, theyl, succinyl, methoxysuccinyl, suberyl, adipyl, azelayl, dansyl, benzyloxycarbonyl, fluorenylmethoxycarbonyl, methoxyazelayl, methoxyadipyl, methoxysuberyl, and 2,4-dinitrophenyl. Many other modifications providing increased potency, prolonged activity, easiness of purification, and/or increased half-life are disclosed in the prior art (WO 02/10195; Villain M et al., 2001).

Preferred alternative, synonymous groups for amino acids derivatives included in peptide mimetics are those defined in Table II. A non-exhaustive list of amino acid derivatives also include aminoisobutyric acid (Aib), hydroxyproline (Hyp), 1,2,3,4-tetrahydro-isoquinoline-3-COOH, indoline-2carboxylic acid, 4-difluoro-proline, L-thiazolidine-4-carboxylic acid, L-homoproline, 3,4-dehydro-proline, 3,4-dihydroxy-phenylalanine, cyclohexyl-glycine, and phenylglycine.

By “amino acid derivative” is intended an amino acid or amino acid-like chemical entity other than one of the 20 genetically encoded naturally occurring amino acids. In particular, the amino acid derivative may contain substituted or non-substituted, linear, branched, or cyclic alkyl moieties, and may include one or more heteroatoms. The amino acid derivatives can be made de novo or obtained from commercial sources (Calbiochem-Novabiochem AG, Switzerland; Bachem, USA).

Various methodologies for incorporating unnatural amino acids derivatives into proteins, using both in vitro and in vivo translation systems, to probe and/or improve protein structure and function are disclosed in the literature (Dougherty D A, 2000). Techniques for the synthesis and the development of peptide mimetics, as well as non-peptide mimetics, are also well known in the art (Golebiowski A et al., 2001; Hruby V J and Balse P M, 2000; Sawyer T K, in “Structure Based Drug Design”, edited by Veerapandian P, Marcel Dekker Inc., pg. 557-663, 1997).

Another object of the present invention are isolated nucleic acids encoding for the polypeptides of the invention having at least one activity of human IFNgamma, the corresponding fusion proteins, or the ligands as disclosed above. Preferably, these nucleic acids should comprise the coding portion of a DNA sequence selected from the group consisting of SEQ ID NO: 1, 3, 5, 7, 9, 11, 13, 15, 17, 19, 21, 23, 25, 27, 29, 31, 33, 35, 37, and 39, or the complement of said DNA sequences. Such coding portions are indicated in FIG. 1-20.

Alternatively, the nucleic acids of the invention are the purified nucleic acids which hybridize under high stringency conditions with a nucleic acid selected from the group consisting of SEQ ID NO: 1, 3, 5, 7, 9, 11, 13, 15, 17, 19, 21, 23, 25, 27, 29, 31, 33, 35, 37, and 39, or a complement of said nucleic acid.

The wording “high stringency conditions” refers to conditions in a hybridization reaction that facilitate the association of very similar molecules and consist in the overnight incubation at 60-65° C. in a solution comprising 50% formamide, 5×SSC (150 mM NaCl, 15 mM trisodium citrate), 50 mM sodium phosphate (pH 7.6), 5× Denhardt's solution, 10% dextran sulphate, and 20 microgram/ml denatured, sheared salmon sperm DNA, followed by washing the filters in O.1×SSC at the same temperature.

These nucleic acids, including nucleotide sequences substantially the same, can be comprised in plasmids, vectors and any other DNA construct which can be used for maintaining, modifying, introducing, or expressing the encoded polypeptide in a cell, in a cell-free expression system, or in a virus. In particular, vectors wherein said nucleic acid molecule is operatively linked to expression control sequences can allow expression in prokaryotic or eukaryotic host cells of the encoded polypeptide.

The wording “nucleotide sequences substantially the same” includes any other nucleic acid sequence that, by virtue of the degeneracy of the genetic code, also encodes for the given amino acid sequences. In this sense, the literature provides indications on preferred or optimized codons for recombinant expression (Kane J F et al., 1995).

The nucleic acids and the vectors can be introduced into cells or virus with different purposes, generating transgenic cells and organisms. For example, a process for producing cells capable of expressing a polypeptide of the invention comprises genetically engineering cells with such vectors or nucleic acids.

In particular, host cells (e.g. bacterial cells) can be modified by transformation for allowing the transient or stable expression of the polypeptides encoded by the nucleic acids and the vectors of the invention. Alternatively, said molecules can be used to generate transgenic animal cells or non-human organisms (by non-/homologous recombination or by any other method allowing their stable in tegration and expression), having enhanced or reduced expression levels of the polypeptides of the invention, when the level is compared with the normal expression levels. Such precise modifications can be obtained by making use of the nucleic acids of the inventions and of technologies associated, for example, to gene therapy (Meth. Enzymol., vol. 346, 2002) or to site-specific recombinases (Kolb A F, 2002). Model systems based on the expression of the polypeptides disclosed in the present patent application can be also generated by gene targeting into human cell lines for the systematic study of their activities (Bunz F, 2002).

The polypeptides of the invention can be prepared by any method known in the art, including recombinant DNA-related technologies, and chemical synthesis technologies. In particular, a method for making a polypeptide of the invention may comprise culturing a host or transgenic cell as described above under conditions in which the nucleic acid or vector is expressed, and recovering the polypeptide encoded by said nucleic acid or vector from cell culture. For example, when the vector expresses the polypeptide as a fusion protein with an extracellular or signal-peptide containing proteins, the recombinant product can be secreted in the extracellular space, and can be more easily collected and purified from cultured cells in view of further processing or, alternatively, the cells can be directly used or administered.

The DNA sequence coding for the proteins of the invention can be inserted and ligated into a suitable episomal or non-/homologously integrating vectors, which can be introduced in the appropriate host cells or virus by any suitable means (transformation, transfection, conjugation, protoplast fusion, electroporation, calcium phosphate-precipitation, direct microinjection, etc.). Factors of importance in selecting a particular plasmid or viral vector include: the ease with which recipient cells that contain the vector, may be recognized and selected from those recipient cells which do not contain the vector; the number of copies of the vector which are desired in a particular host; and whether it is desirable to be able to “shuttle” the vector between host cells of different species.

The vectors should allow the expression of the isolated or fusion protein including the polypeptide of the invention in the Prokaryotic or Eukaryotic host cells under the control of transcriptional initiation/termination regulatory sequences, which are chosen to be inducible or constitutively active in said cell. A cell line substantially enriched in such cells can be then isolated to provide a stable cell line.

Different transcriptional and translational regulatory sequences may be employed for Eukaryotic hosts, depending on the nature of the host (e.g. yeasts, insect, plant, or mammalian cells). They may be derived form viral sources, such as adenovirus, bovine papilloma virus, Simian virus or the like, where the regulatory signals are associated with a particular gene which has a high level of expression. Examples are the TK promoter of the Herpes virus, the SV40 early promoter, the yeast gal4 gene promoter, etc. Transcriptional initiation regulatory signals may be selected which allow for repression and activation, so that expression of the genes can be modulated. The cells stably transformed by the introduced DNA can be selected by introducing one or more markers allowing the selection of host cells that contain the expression vector. The marker may also provide for phototrophy to an auxotropic host, resistance to biocides (e.g. antibiotics) or to heavy metals (e.g. copper). The selectable marker gene can either be directly linked to the DNA sequences to be expressed in the same vector, or introduced into the same cell by co-transfecting another vector.

Host cells may be either prokaryotic or eukaryotic. Preferred are eukaryotic hosts, e.g. mammalian cells, such as human, monkey, mouse, and Chinese Hamster Ovary (CHO) cells, because they provide post-translational modifications to proteins, including correct folding and glycosylation. Also yeast cells can carry out post-translational peptide modifications including glycosylation. A number of recombinant DNA strategies exist which utilize strong promoter sequences and high copy number of plasmids that can be utilized for production of the desired proteins in yeast, which recognizes leader sequences in cloned mammalian gene products and secretes peptides bearing leader sequences (i.e., pre-peptides).

The above mentioned embodiments of the invention can be achieved by combining the disclosure provided by the present patent application on the sequence of novel polypeptides with the knowledge of common molecular biology techniques.

Many books and reviews provides teachings on how to clone and produce recombinant proteins using vectors and Prokaryotic or Eukaryotic host cells, such as some titles in the series “A Practical Approach” published by Oxford University Press (“DNA Cloning 2: Expression Systems”, 1995; “DNA Cloning 4: Mammalian Systems”, 1996; “Protein Expression”, 1999; “Protein Purification Techniques”, 2001).

Moreover, literature also provides an overview of the technologies for expressing polypeptides in a high-throughput manner (Chambers S P, 2002; Coleman T A et al., 1997), of the cell systems and the processes used industrially for the large-scale production of recombinant proteins having therapeutic applications (Andersen D C and Krummen L, 2002, Chu L and Robinson D K, 2001), and of alternative eukaryotic expression systems for expressing the polypeptide of interest, which may have considerable potential for the economic production of the desired protein, such the ones based on transgenic plants (Giddings G, 2001) or the yeast Pichia pastoris (Lin Cereghino G P et al., 2002). Recombinant protein products can be rapidly monitored with various analytical technologies during purification to verify the amount and the quantity of the expressed polypeptides (Baker K N et al., 2002), as well as to check properties like bioequivalence and immunogenicity (Schellekens H, 2002; Gendel S M, 2002).

Totally synthetic proteins are disclosed in the literature (Brown A et al., 1996), and many examples of chemical synthesis technologies, which can be effectively applied for the polypeptides of the invention given their short length, are available in the literature, as solid phase or liquid phase synthesis technologies. For example, the amino acid corresponding to the carboxy-terminus of the peptide to be synthetized is bound to a support which is insoluble in organic solvents, and by alternate repetition of reactions, one wherein amino acids with their amino groups and side chain functional groups protected with appropriate protective groups are condensed one by one in order from the carboxy-terminus to the amino-terminus, and one where the amino acids bound to the resin or the protective group of the amino groups of the peptides are released, the peptide chain is thus extended in this manner. Solid phase synthesis methods are largely classified by the tBoc method and the Fmoc method, depending on the type of protective group used. Typically used protective groups include tBoc (t-butoxycarbonyl), Cl-Z (2-chlorobenzyloxycarbonyl), Br-Z (2-bromobenzyloxycarbonyl), Bzl (benzyl), Fmoc (9-fluorenylmethoxycarbonyl), Mbh (4,4′-dimethoxydibenzhydryl), Mtr (4-methoxy-2,3,6-trimethylbenzenesulphonyl), Trt (trityl), Tos (tosyl), Z (benzyloxycarbonyl) and Cl2-Bzl (2,6-dichlorobenzyl) for the amino groups; NO2 (nitro) and Pmc(2,2,5,7,8-pentamethylchromane-6-sulphonyl) for the guanidino groups); and tBu (t-butyl) for the hydroxyl groups). After synthesis of the desired peptide, it is subjected to the de protection reaction and cut out from the solid support. Such peptide cutting reaction may be carried with hydrogen fluoride or tri-fluoromethane sulfonic acid for the Boc method, and with TFA for the Fmoc method.

The purification of the polypeptides of the invention can be carried out by any one of the methods known for this purpose, i.e. any conventional procedure involving extraction, precipitation, chromatography, electrophoresis, or the like. A further purification procedure that may be used in preference for purifying the protein of the invention is affinity chromatography using monoclonal antibodies or affinity groups, which bind the target protein and which are produced and immobilized on a gel matrix contained within a column. Impure preparations containing the proteins are passed through the column. The protein will be bound to the column by heparin or by the specific antibody while the impurities will pass through. After washing, the protein is eluted from the gel by a change in pH or ionic strength. Alternatively, HPLC (High Performance Liquid Chromatography) can be used. The elution can be carried using a water-acetonitrile-based solvent commonly employed for protein purification.

The disclosure of the novel polypeptides of the invention, and the reagents disclosed in connection to them (antibodies, nucleic acids, cells) allows also to screen and characterize compounds (proteins, as well as small organic molecules) that are capable to enhance or reduce their expression level into a cell or in an animal. Examples of compounds that can reduce or block the expression of polypeptides are antisense oligonucleotides (Stein C A, 2001) or small interfering, double stranded RNA molecules that can trigger RNA interference-mediated silencing (Paddison P J et al., 2002; Lewis D L et al., 2002). These compounds are intended as antagonists (in addition to the ones above described in connection to mutants and ligands) in the context of the possible mechanism of antagonism for blocking cytokine-controlled pathways as defined in the literature (Choy E H and Panayl G S. 2001; Dower S K, 2000).

“Oligonucleotides” refers to either a single stranded polydeoxynucleotide or two complementary polydeoxynucleotide strands that may be chemically synthesized. Such synthetic oligonucleotides have no 5′ phosphate and thus will not ligate to another oligonucleotide without adding a phosphate with an ATP in the presence of a kinase. A synthetic oligonucleotide will ligate to a fragment that has not been dephosphorylated.

The invention includes purified preparations of the products of the invention (polypeptides, nucleic acids, cells, ligands, peptide mimetics). Purified preparations, as used herein, refers to the preparations which containing at least 1%, preferably at least 5%, by dry weight of the compounds of the invention.

The present patent application discloses a series of novel polypeptides and of related reagents having one or more human IFNgamma-related activities that can be exploited for several possible applications. In particular, whenever the increase of a human IFNgamma-related activity of a polypeptide of the invention is desirable in the therapy or in the prevention of a disease, reagents such as the disclosed polypeptides having a defined homology with the consensus sequence pIFNFHcon, the corresponding fusion proteins and peptide mimetics, the encoding nucleic acids, the expressing cells, or the compounds enhancing their expression can be used.

Therefore, the present invention discloses pharmaceutical compositions for the treatment or prevention of diseases needing an increase in a human IFNgamma activity of a polypeptide of the invention, which contain one of the disclosed polypeptides having a defined homology with the consensus sequence pIFNFHcon, the corresponding fusion proteins and peptide mimetics, the encoding nucleic acids, the expressing cells, or the compounds enhancing their expression, as active ingredient. The process for the preparation of these pharmaceutical compositions comprises combining the disclosed polypeptides having a defined homology with the consensus sequence pIFNFHcon, the corresponding fusion proteins and peptide mimetics, the encoding nucleic acids, the expressing cells, or the compounds enhancing their expression, together with a pharmaceutically acceptable carrier. Methods for the treatment or prevention of diseases needing an increase in a human IFNgamma activity of a polypeptide of the invention, comprise the administration of a therapeutically effective amount of the disclosed INSP037 like polypeptides, the corresponding fusion proteins and peptide mimetics, the encoding nucleic acids, the expressing cells, or the compounds enhancing their expression.

Amongst the novel molecules disclosed in the present patent application, the ligands or the compounds reducing the expression or the activity of polypeptides of the invention have several applications, and in particular they can be used in the therapy or in the diagnosis of a disease associated to the excessive human IFNgamma activity of a polypeptide of the invention.

Therefore, the present invention discloses pharmaceutical compositions for the treatment or prevention of diseases associated to the excessive human IFNgamma activity of a polypeptide of the invention, which contain one of the ligands or compounds reducing the expression or the activity of such polypeptides, as active ingredient. The process for the preparation of these pharmaceutical compositions comprises combining the ligand or the compound, together with a pharmaceutically acceptable carrier. Methods for the treatment or prevention of diseases associated to the excessive IFNgamma-related activity of the polypeptide of the invention, comprise the administration of a therapeutically effective amount of the antagonist, the ligand or of the compound.

The present patent application discloses novel polypeptides having a defined homology with the consensus sequence pIFNFHcon and a series of related reagents that may be useful, as active ingredients in pharmaceutical compositions appropriately formulated, in the treatment or prevention of diseases for which a compound having a human IFNgamma-related activity, or its antagonist and inhibitor, may provide beneficial effects, such as cell proliferative disorders, autoimmune/inflammatory disorders, cardiovascular disorders, neurological disorders, or bacterial and viral infections. A non-exhaustive lists of disorders include multiple sclerosis, graft-vs-host disease, lymphomas, leukaemia, Crohn's disease, asthma, septic shock, type I and type II diabetes, allergies, asthma, psoriasis, inflammatory bowel disease, ulcerative colitis, fibrotic diseases, rheumatoid arthritis, and neuroblastoma.

The therapeutic applications of the polypeptides of the invention and of the related reagents can be evaluated (in terms or safety, pharmacokinetics and efficacy) by the means of the in vivo/in vitro assays making use of animal cell, tissues and models developed for human IFNgamma and/or IFNgamma binding proteins (Boehm U et al., 1997; Bach E A et al., 1997), including their orthologs or antagonists, or by the means of in silico/computational approaches (Johnson D E and Wolfgang G H, 2000), known for the validation of IFNs and other biological products during drug discovery and preclinical development.

It is intended that any disclosed use or activity related to human IFNgamma (or its orthologs or antagonists) disclosed in the prior art can be also applicable to any corresponding embodiment of the present invention, such as therapeutic uses and compositions, alone or in combination with another compounds (EP311616, WO 01/34180, EP 490250; EP203580; EP502997; EP886527; EP696639; Ziesche R et al., 1999; WO 01/34180; EP203580; WO 90/03189; WO 95/22328; U.S. Pat. No. 5,170,591; WO 02/102312; U.S. Pat. No. 5,666,312; WO 91/07984; U.S. Pat. No. 5,198,212; EP1265996; U.S. Pat. No. 6,036,956; EP 1140990; WO 98/28001; WO 94/12531; WO 94/14497; WO 02/98460; WO 99/09055, WO 00/32634), formulations (EP697887, WO 01/36001), expression systems (WO 01/57218) known for human IFNgamma.

The pharmaceutical compositions of the invention may contain, in addition to polypeptides having a defined homology with the consensus sequence pIFNFHcon or to the related reagent, suitable pharmaceutically acceptable carriers, biologically compatible vehicles and additives which are suitable for administration to an animal (for example, physiological saline) and eventually comprising auxiliaries (like excipients, stabilizers, adjuvants, or diluents) which facilitate the processing of the active compound into preparations which can be used pharmaceutically.

The pharmaceutical compositions may be formulated in any acceptable way to meet the needs of the mode of administration. For example, of biomaterials, sugar-macromolecule conjugates, hydrogels, polyethylene glycol and other natural or synthetic polymers can be used for improving the active ingredients in terms of drug delivery efficacy. Technologies and models to validate a specific mode of administration and delivery are disclosed in literature in general (Davis B G and Robinson M A, 2002; Gupta P et al., 2002; Luo B and Prestwich G D, 2001; Cleland J L et al., 2001; Pillal O and Panchagnula R, 2001), as well as specifically for IFNgamma (Younes H M and Amsden B G, 2002).

Polymers suitable for these purposes are biocompatible, namely, they are non-toxic to biological systems, and many such polymers are known. Such polymers may be hydrophobic or hydrophilic in nature, biodegradable, non-biodegradable, or a combination thereof. These polymers include natural polymers (such as collagen, gelatin, cellulose, hyaluronic acid), as well as synthetic polymers (such as polyesters, polyorthoesters, polyanhydrides). Examples of hydrophobic non-degradable polymers include polydimethyl siloxanes, polyurethanes, polytetrafluoroethylenes, polyethylenes, polyvinyl chlorides, and polymethyl methaerylates. Examples of hydrophilic non-degradable polymers include poly(2-hydroxyethyl methacrylate), polyvinyl alcohol, poly(N-vinyl pyrrolidone), polyalkylenes, polyacrylamide, and copolymers thereof. Preferred polymers comprise as a sequential repeat unit ethylene oxide, such as polyethylene glycol (PEG).

Any accepted mode of administration can be used and determined by those skilled in the art to establish the desired blood levels of the active ingredients. For example, administration may be by various parenteral routes such as subcutaneous, intravenous, intradermal, intramuscular, intraperitoneal, intranasal, transdermal, oral, or buccal routes. The pharmaceutical compositions of the present invention can also be administered in sustained or controlled release dosage forms, including depot injections, osmotic pumps, and the like, for the prolonged administration of the polypeptide at a predetermined rate, preferably in unit dosage forms suitable for single administration of precise dosages.

Parenteral administration can be by bolus injection or by gradual perfusion over time. Preparations for parenteral administration include sterile aqueous or non-aqueous solutions, suspensions, and emulsions, which may contain auxiliary agents or excipients known in the art, and can be prepared according to routine methods. In addition, suspension of the active compounds as appropriate oily injection suspensions may be administered. Suitable lipophilic solvents or vehicles include fatty oils, for example, sesame oil, or synthetic fatty acid esters, for example, sesame oil, or synthetic fatty acid esters, for example, ethyl oleate or triglycerides. Aqueous injection suspensions that may contain substances increasing the viscosity of the suspension include, for example, sodium carboxymethyl cellulose, sorbitol, and/or dextran. Optionally, the suspension may also contain stabilizers. Pharmaceutical compositions include suitable solutions for administration by injection, and contain from about 0.01 to 99.99 percent, preferably from about 20 to 75 percent of active compound together with the excipient.

The wording “therapeutically effective amount” refers to an amount of the active ingredients that is sufficient to affect the course and the severity of the disease, leading to the reduction or remission of such pathology. The effective amount will depend on the route of administration and the condition of the patient.

The wording “pharmaceutically acceptable” is meant to encompass any carrier, which does not interfere with the effectiveness of the biological activity of the active ingredient and that is not toxic to the host to which is administered. For example, for parenteral administration, the above active ingredients may be formulated in unit dosage form for injection in vehicles such as saline, dextrose solution, serum albumin and Ringer's solution. Carriers can be selected also from starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, ethanol, and the various oils, including those of petroleum, animal, vegetable or synthetic origin (peanut oil, soybean oil, mineral oil, sesame oil).

It is understood that the dosage administered will be dependent upon the age, sex, health, and weight of the recipient, kind of concurrent treatment, if any, frequency of treatment, gravity of the disease, and the nature of the effect desired. The dosage will be tailored to the individual subject, as is understood and determinable by one of skill in the art. The total dose required for each treatment may be administered by multiple doses or in a single dose. The pharmaceutical composition of the present invention may be administered alone or in conjunction with other therapeutics directed to the condition, or directed to other symptoms of the condition. Usually a daily dosage of active ingredient is comprised between 0.01 to 100 milligrams per kilogram of body weight per day. Ordinarily 1 to 40 milligrams per kilogram per day given in divided doses or in sustained release form is effective to obtain the desired results. Second or subsequent administrations can be performed at a dosage, which is the same, less than, or greater than the initial or previous dose administered to the individual.

Apart from the methods having a therapeutic or a production purpose, several other applications can make use of the polypeptides having a defined homology with the consensus sequence pIFNFHcon and of the related reagents disclosed in the present patent application.

In a first example, a method for screening candidate compounds effective to treat a disease related to a polypeptides of the invention having a defined homology with the consensus sequence pIFNFHcon, comprises:
(a) contacting a cell expressing such polypeptide, transgenic non-human animals, or transgenic animal cells having enhanced or reduced expression levels of the polypeptide, with a candidate compound and
(b) determining the effect of the compound on the animal or on the cell.

In a second example, a method for identifying a candidate compound as an antagonist/inhibitor or agonist/activator of a polypeptide of the invention having a defined homology with the consensus sequence pIFNFHcon, comprises:
(a) contacting the polypeptide and the compound with a mammalian cell or a mammalian cell membrane; and
(b) measuring whether the compound blocks or enhances the interaction of the polypeptide, or the response that results from such interaction, with the mammalian cell or the mammalian cell membrane.

In a third example, methods for determining the activity and/or the presence of the peptide of the invention having a defined homology with the consensus sequence pIFNFHcon in a sample, can detect either the polypeptide or the encoding RNA/DNA. Thus, such a method comprises:
(a) providing a protein-containing sample;
(b) contacting said sample with a ligand of the invention; and
(c) determining the presence of said ligand bound to said polypeptide, thereby determining the activity and/or the presence of polypeptide in said sample.

Alternatively, the method comprises:
(a) providing a nucleic acids-containing sample;
(b) contacting said sample with a nucleic acid of the invention; and
(c) determining the hybridization of said nucleic acid with a nucleic acid into the sample, thereby determining the presence of the nucleic acid in the sample.

In this sense, primer sequences containing the sequences SEQ ID NO: 41-78 (Table III) can be used as well for determining the presence or the amount of a transcript or of a nucleic acid encoding a polypeptide of invention having a defined homology with the consensus sequence pIFNFHcon in a sample by means of Polymerase Chain Reaction amplification, nucleic acid sequencing, or nucleic acid hybridization.

A further object of the present invention are kits for measuring the activity and/or the presence of a polypeptide of the invention having a defined homology with the consensus sequence pIFNFHcon in a sample, comprising one or more of the reagents disclosed in the present patent application: a polypeptide of the invention having a defined homology with the consensus sequence pIFNFHcon, a ligand, their active conjugates or complexes, an isolated nucleic acid or vector, a pharmaceutical composition, an expressing cell, a compound increasing or decreasing the expression levels, and/or primer sequences containing any of the sequences SEQ ID NO: 41-78.

Those kits can be used for in vitro diagnostic or screenings methods, and their actual composition should be adapted to the specific format of the sample (e.g. biological sample tissue from a patient), and the molecular species to be measured. For example, if it is desired to measure the concentration of the INSP037-like polypeptide, the kit may contain an antibody and the corresponding protein in a purified form to compare the signal obtained in Western blot. Alternatively, if it is desired to measure the concentration of the transcript for the polypeptide of the invention having a defined homology with the consensus sequence pIFNFHcon, the kit may contain a specific nucleic acid probe designed on the corresponding ORF sequence, or may be in the form of nucleic acid array containing such probe, or the primer sequences disclosed as SEQ ID NO: 41-78 (Table III). The kits can be also in the form of protein- or cell-based microarrays (Templin M F et al., 2002; Pellois J P et al., 2002; Blagoev B and Pandey A, 2001), allowing high-throughput proteomics studies, by making use of the proteins, peptide mimetics and cells disclosed in the present patent application.

Finally, given that some of the polypeptides of the invention having a defined homology with the consensus sequence pIFNFHcon have shown a particularly effective secretion without the addition of any heterologous signal sequence (Martoglio B and Dobberstein B, 1998), such polypeptides, or any secreted fragment, can be used as signal sequences.

All publications, patents and patent applications cited herein are incorporated in full by reference for any purpose.

The invention will now be described with reference to the specific embodiments by means of the following Examples, which should not be construed as in any way limiting the present invention. The content of the description comprises all modifications and substitutions which can be practiced by a person skilled in the art in light of the above teachings and, therefore, without extending beyond the meaning and purpose of the claims.

### EXAMPLES

### Example 1

### Selection of Open Reading Frames (ORFs) Encoding for Polypeptides Homologous to INSP037, Called pIFNFHs

INSP037 was identified as an IFNgamma-like protein encoded by an ORF in human genome (GB patent application No. 0130720.6). The sequence of this ORF was used to search for homologous ORFs in human genome (Celera and GenBank databases). The homology was detected using the BLAST (Basic Local Alignment Search Tool; NCBI version 2), an algorithm which generates local alignments between a query and a hit sequence (Gish W and States D J, 1993; Pearson W R and Miller W, 1992; Altschul S F et al., 1990). In this case the TBLASTN algorithm was used with the INSP037 protein sequence as a query. TBLASTN compares the query sequence to the database translated into 6 frames and can therefore identify a protein match to a DNA sequence in any reading frame. BLAST parameters used were: Comparison matrix=BLOSUM62; word length=3; .E value cutoff=10; Gap opening and extension=default; No filter.

The pattern of the homologous regions were extracted from the BLAST output file using a script written in PERL (Practical Extraction and Report Language), a programming language having powerful pattern matching functions into large text data files allowing the extraction of information from genomic DNA sequences, starting from an alpha-numerical expression describing a defined consensus sequence (Stein L D, 2001). Another PERL script was used to retrieve the entire ORFs having such INSP037-like features, extending the sequence 6 to the first potential start methionine and 3′ to the first stop codon.

A total of 20 ORFs out of the 93 hits matching the original query generated on the basis of INSP037 protein sequence were selected since they have a start Methionine and a stop codon separated by between 75 and 150 codons. IFNFHs selected DNA sequences (SEQ ID NO: 1, 3, 5, 7, 9, 11, 13, 15, 17, 19, 21, 23, 25, 27, 29, 31, 33, 35, 37, and 39), belong to different human chromosomes, potentially encode for protein sequences (SEQ ID NO: 2, 4, 6, 8, 10, 12, 14, 16, 18, 20, 22, 24, 26, 28, 30, 32, 34, 36, 38, and 40) having a significant homology with INSP037 (BLAST E value minor or equal to 7e⁻²³), with level of identity composed between 67% and 78.5% (FIGS. 1-20). The novelty of the protein sequences was assessed by searching protein databases (SwissProt/Trembl and Derwent GENESEQ) using BLAST.

Amongst these sequences characterized as novel INSP0374-like polypeptides, three of them (pIFNFH04, pIFNFH32, and pIFNFH20) are less than 10% longer than INSP037, while all the other sequences more than 10% longer due to an extended C-terminal region (pIFNFH08, pIFNFH12, pIFNFH25, pIFNFH36, pIFNFH37, pIFNFH23, pIFNFH27, pIFNFH14, pIFNFH01, pIFNFH10, pIFNFH11, pIFNFH13, pIFNFH31, pIFNFH03, and pIFNFH15), or to extended N-terminal and C-terminal regions (pIFNFH39 and pIFNFH42). The extended C-terminal regions present some significant local homologies amongst the different IFNFHs (FIG. 21). Even if not identified in FIGS. 1-20, at least some of the selected polypeptides contain a functional signal peptide (Example 3).

INSP037 and pIFNFHs can be aligned comparing the conservation of the different residues. This alignment leads to the identification of a consensus sequence, called pIFNFHcon, which includes 75 amino acids, and in particular ten positions (Ala10, Gly12, Arg26, Ala31, Lys35, Phe47, Gln55, Glu57, Lys63, and Ile75) that are specific for pIFNFHs and not conserved in INSP037 (FIG. 21). All these residues are mutated in a non-conservative manner in INSP037.

According to the homology to the consensus sequence pIFNFHcon can be divided in three groups.

A first group of pIFNFHs includes polypeptides that comprise a sequence having at least 80% of homology with the complete sequence of pIFNFHcon and no non-conservative mutations in the positions corresponding to Ala10, Gly12, Arg26, Ala31, Lys35, Phe47, Gln55, Glu57, Lys63, and Ile75 in pIFNFHcon. Examples of such sequences are pIFNFH15, pIFNFH32, and pIFNFH37.

A second group of pIFNFHs includes polypeptides that comprise a sequence having at least 80% of homology with the complete sequence of pIFNFHcon and one or two non-conservative mutations in the positions corresponding to Ala10, Gly12, Arg26, Ala31, Lys35, Phe47, Gln55, Glu57, Lys63, and Ile75 in pIFNFHcon. Examples of such sequences are pIFNFH04, pIFNFH03, pIFNFH08, pIFNFH20, pIFNFH23, pIFNFH12, pIFNFH25, pIFNFH13, pIFNFH14, pIFNFH36, and pIFNFH39.

A third group of pIFNFHs includes polypeptides that comprise a sequence having at least 80% of homology with the complete sequence of pIFNFHcon and three, four, or five non-conservative mutations in the positions corresponding to Ala10, Gly12, Arg26, Ala31, Lys35, Phe47, Gln55, Glu57, Lys63, and Ile75 in pIFNFHcon. Examples of such sequences are pIFNFH11, pIFNFH27, pIFNFH01, pIFNFH31, pIFNFH10, and pIFNFH42.

The consensus sequence pIFNFHcon, in connection with the identification of specific residues to be conserved, characterizes pIFNFHs and allows to make a clear distinction not only between pIFNFHs and INSP037 but also between pIFNFHs and sequences disclosed in the literature that are homologous to a portion of INSP037 and pIFNFHs, and identified as protein #134 (Derwent DGENE database ace. No. ABG00143; SEQ ID NO: 157; WO 01/75067) and SEQ ID NO: 30734 (Derwent DGENE database ace. No. AAM70428; SEQ ID NO: 158; WO 01/57276).

The C-terminal segment of the first one of these sequences overlaps with N-terminal and central portion of pIFNFHcon, without including the C-terminal portion containing four of the ten conserved residues in pIFNFHs. The N-terminal segment of the second one of these sequences overlaps with C-terminal and central portion of pIFNFHcon, without including the N-terminal portion containing three of the ten conserved residues in pIFNFHs (FIG. 22). Therefore, none of these sequences discloses pIFNFHcon, neither provides an indication of the specific residues conserved in such consensus sequence.

### Example 2

### Cloning of the IFNFHs Nucleic Acid Sequences from Human Genomic DNA

The selected IFNFH nucleic acid sequences, each corresponding to a single axon, were cloned (with the exception of IFNFH25) from human genomic DNA into a cloning vector, and then transferred into an expression vector using Polymerase Chain Reaction (PCR), with pairs of forward/reverse primers specific for each ORF (see arrows in FIGS. 1-12 and 14-20).

The cloning primers (CL series; SEQ ID NO: 41-78, Table III), containing from 21 to 30 nucleotides, were designed for amplifying each ORF using human genomic DNA as template, since all ORFs are uninterrupted on human chromosomes. The forward primers start from three nucleotides before initial ATG. The reverse primers are complementary to the 3′ end of the ORF, including the stop codon. Being the N-terminal sequences very similar amongst the different IFNFHs, the reverse primers actually are actually responsible for the specificity of the amplification react on.

The PCR was performed by mixing the following components in each ORF-specific reaction (total volume of 50 μl in double-distilled water):
150 ng human genomic DNA (Clontech)
1.2 μM primers (0.6 μM each primer)
240 μM dNTP (Invitrogen)
0.5 μl AmpliTaq (2.5 Units; Applied Biosystems)
5 AmpliTaq buffer 10× (Applied Biosystems)

The PCR reactions were performed using an initial denaturing step if 94° C. for 2 minutes, followed by 30 cycles:
94° C. for 30 seconds
55° C. for 30 seconds
72° C. for 30 seconds

After a final elongation step of 72° C. for 10 minutes, the PCR products were directly subcloned into the pCRII-TOPO vector using the TOPO™ cloning system (Invitrogen), according to manufacturer's standard protocol. The TOPO cloning system is a variation of the TA cloning system allowing the rapid cloning of PCR products, taking advantage from the fact that Taq polymerase leaves a single Adenosine at the 3′ end of PCR products. Since the TOPO vector has single-stranded Thymine overhangs, Topoisomerase I enzyme is able to join the T-ends of the vector to the A-overhangs of the PCR product, which can be used without any purification step.

The resulting plasmids (pCRTOPO-ORF series) were used to transform E. coli cells (TOP10F′, Invitrogen, supplied with the TOPO TA Cloning Kit), obtaining several clones for each ORF. Plasmid DNA was isolated using a commercial kit (WIZARD Plasmid Minipreps: Promega) and sequenced to verify the identity of the amplified and cloned sequence with the originally selected human genomic DNA sequence.

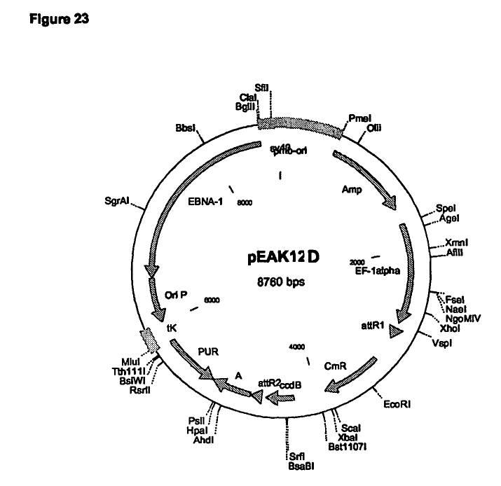

The plasmids containing the desired sequences were used in a further round of PCR reactions necessary for transferring the ORFs into the expression vector pEAK12D (FIG. 23), which allows the expression of the cloned insert under the control of EF-1α promoter and in frame with a 6-His Tag sequence, using the Gateway cloning system (Invitrogen).

The expression vector pEAK12D was constructed by modifying pEAK12 (Edge Biosystems). This vector was digested with HindIII and NotI, made blunt ended with Klenow and dephosphorylated using calf-intestinal alkaline phosphatase. After dephosphorylation, the vector was ligated to blunt ended Gateway reading frame cassette C (Gateway vector conversion system, Invitrogen cat no. 11828-019) which contains AttR recombination sites flanking the ccdB gene (marker for negative selction of non-recombinant plasmids) and chloramphenicol resistance. The resulting plasmids were used to transform DB3.1 E. coli cells, which allow propagation of vectors containing the ccdB gene. Miniprep DNA was isolated from several of the resultant colonies and digested with AseI/EcoRI to identify clones yielding a 670 bp fragment, obtainable only when the cassette had been inserted in the correct orientation. The resultant plasmid was called pEAK12D.

Two series of primers were designed to add the ATTB1 and ATTB2 recombination sites (necessary for the integration in the expression vector) at the 5′ and 3′ end, respectively, of the ORF-containing insert in the first series of primers (EX1 series; SEQ ID NO: 79-116, Table IV), the original ORF-specific CL primers were modified by adding, at the 5′ end, the sequence AAGCAGGCTTCGCCACC (for forward primers) or GTGATGGTGATGGTG (for reverse primers, but after eliminating the nucleotides complementary to the stop codon). In the second series of primers (EX2 series; SEQ ID NO: 117-454, Table V), the original ORF-specific CL primers were modified by adding, at the 5′ end, the sequence GGGGACAAGTTTGTACAAAAAAGC AGGCTTCGCCACC (for forward primers) or GGGGACCACTTTGTACAAGAAAGCTG GGTTTCAATGGTGATGGTGATGGTG (for reverse primers, but after eliminating the nucleotides complementary to the stop codon). These reverse primers contain the codons for the 6-His tag, then resulting fused in frame with the ORFs at their C-terminal end.

The PCR amplification was performed in 2 consecutive reactions. The first one was performed by mixing the following components (total volume 50 μl in double-distilled water):
25 ng pCRTOPO-ORF vector
5 mM dNTP (Invitrogen)
0.5 μl Pfx DNA polymerase (Invitrogen)
0.5 μl each EX1 primer (100 μM)
5 μl 10×Pfx polymerase buffer (Invitrogen)

The PCR reactions were performed using an initial denaturing step of 95° C. for 2 minutes, followed by 10 cycles:
94° C. for 15 seconds
68° C. for 30 seconds

The PCR products were purified using the Wizard PCR prep DNA purification system (Promega), and added as templates in a second PCR reaction including the following components (total volume 50 μl in double-distilled water):
10 μl purified PCR product
5 mM dNTP (Invitrogen)
0.5 μl Pfx DNA polymerase (Invitrogen)
0.5 μl each EX2 primer (100 μM)
5 μl 10×Pfx polymerase buffer (Invitrogen)

The PCR reactions were performed an initial denaturing step of 95° C. for 1 minute, followed by 4 cycles:
94° C. for 15 seconds
50° C. for 30 seconds
68° C. for 3 minutes 30 seconds

Then the following conditions were applied for 25 cycles:
94° C. for 15 seconds
55° C. for 30 seconds
68° C. for 3 minutes 30 seconds.

The DNA fragments resulting from the PCR reactions were purified as described before and recombined into the pEAK12D vector using the Gateway system.

First, the following 10 μl reactions were assembled:

| | | |
|---|---|---|
| pDONR-201 (0.1 μg/μl) | 1.5 | μl |
| PCR product | 5 | μl |
| BP buffer | 2 | μl |
| BP enzyme mix | 1.5 | μl |

After being incubated at room temperature for 1 hour, the reaction was stopped by adding proteinase K (1 μl, 2 μg) and incubating at 37° C. for further 10 minutes.

An aliquot of this reaction (2 μl) was used for transforming E. coli cells (strain DH10B) by electroporation. Plasmid DNA was prepared for 4 clones for each ORF and used for parallel 10 μl recombination reactions containing:

| | | |
|---|---|---|
| pEAK12D (0.1 μg/μl) | 1.5 | μl |
| Plasmid DNA | 1.5 | μl |
| ddH20 | 3.5 | μl |
| LR buffer | 2 | μl |
| LR enzyme mix | 1.5 | μl |

After being incubated at room temperature for 1 hour, the reaction was stopped by adding proteinase K (1 μl, 2 μg) and incubating at 37° C. for further 10 minutes. An aliquot of this reaction (1 μl) was used for transforming DH10B E. coli cells by electroporation. The clones containing the correct insert were identified first by performing colony PCR on 3 colonies using the forward and reverse vector primers pEAK12D F1 (GCCAGCTTGGCACTTGATGT) and pEAK12D R1 (GATGGAGGTGGA CGTGTCAG), then confirmed by sequencing the insert with the same primer.

### Example 3

### Expression and Purification of the His-Tagged pIFNFHs Polypeptides in Mammalian Cells

The vectors generated in Example 2 were used to express pIFNFHs in Human Embryonic Kidney cells expressing the Epstein-Barr virus Nuclear Antigen (cell line HEK293-EBNA).

The cells were seeded in T225 flasks (50 ml at a density of 2×10⁵ cells/ml) from 16 to 20 hours prior to transfection, which was performed using the cationic polymer reagent JetPEI™ (PolyPlus-transfection; 2 μl/μg of plasmid DNA). For each flask, 113 μg of the ORF-specific pEAK12D plasmid, which were prepared using CsCl (Sambrook, J et al. “Molecular Cloning, a laboratory manual”; 2nd edition. 1989; Cold Spring Harbor Laboratory Press), were co-transfected with 2.3 μg of a plasmid acting as positive control since it expresses Green Fluorescent Protein (GFP) in a constitutive manner. The plasmids, diluted in 230 μl of JetPEI™ solution, were added to 4.6 ml of NaCl 150 mM, vortexed and incubated for 30 minutes at room temperature. This transfection mix was then added to the T225 flask and incubated at 37° C. for 6 days. An aliquot of the cultures was then exposed to UV irradiation to check the transfection efficiency by evaluating GFP fluorescence.

Culture medium from HEK293-EBNA cells transfected with the ORF-specific pEAK12D plasmids were pooled and 100 ml of the medium were diluted to 200 ml with 100 ml of ice-cold buffer A (50 mM NaH₂PO₄; 600 mM NaCl; 8.7% (w/v) glycerol, pH 7.5), which is the same buffer used for equilibrating the affinity column on which His-tagged proteins were subsequently immobilized and eluted. The solution was filtered through a 0.22 μm sterile filter (Millipore) and kept at 4° C. in 250 ml sterile square media bottles until further processing.

Two consecutive chromatography procedures were applied to the samples using an HPLC-based system (Perfusion Chromatography™, PerSeptive Biosystems) including a VISION workstation (BioCAD™ series), POROS™ chromatographic media, and an external 250 ml-sample loader (Labomatic), all kept at 4° C.

In the first chromatography step, a Ni-metal affinity column (0.83 ml, POROS 20 MC) was first regenerated with 30 column volumes of EDTA solution (100 mM EDTA; 1 M NaCl; pH 8.0), and then recharged with Ni ions through washing with 15 column volumes of the Ni solution (100 mM NiSO₄). The column is subsequently washed with 10 column volumes of buffer A, 7 column volumes of buffer B (50 mM NaH₂PO₄; 600 mM NaCl; 8.7% (w/v) glycerol, 400 mM; imidazole, pH 7.5), and finally equilibrated with 15 column volumes of buffer A containing 15 mM imidazole. The sample loader charged the protein-containing solution onto the Ni metal affinity column at a flow rate of 10 ml/min. The column was then washed with 12 column volumes of Buffer A, followed by 28 column volumes of Buffer A containing a concentration of imidazole (20 mM) allowing the elution of contaminating proteins that are loosely attached to the Ni-column. The His-tagged protein is finally eluted with 10 column volumes of Buffer B at a flow rate of 2 ml/min, collecting collected 1.6 ml fractions.

In the second chromatography step, a gel-filtration column (10 ml G-25 Sephadex) was regenerated with 2 ml of buffer D (137 mM NaCl; 2.7 mM KCl; 1.5 mM KH₂PO₄; 8 mM Na₂HPO₄; 1 M NaCl; pH 7.2), and then equilibrated with 2 column volumes of buffer C (137 mM NaCl; 2.7 mM KCl; 1.5 mM KH₂PO₄; 8 mM Na₂HPO₄; 20% (w/v) glycerol; pH 7.4) before injecting the Ni-column peak fractions onto this column. The sample is eluted with buffer C and the desalted sample is recovered in 2.2 ml fractions.

The peak fractions from the gel-filtration column were then analyzed for their protein content using SDS-PAGE and the parallel detection by Coomassie staining and by Western blot with antibodies recognizing His-tags.

The fractions were filtered through a 0.22 μm sterile centrifugation filter (Millipore) and aliquots (20 μl) were analyzed on SDS-PAGE (4-12% NuPAGE gel; Novex). Protein concentrations were determined in the samples that show detectable protein bands by Coomassie staining, using the BCA Protein Assay kit (Pierce) and Bovine Serum Albumin as standard. The gel for the Western blot analysis was electrotransferred to a nitrocellulose membrane at 290 mA at 4° C. for 1 hour. The membrane was blocked with 5% milk powder in PBS (137 mM NaCl; 2.7 mM KCl; 1.5 mM KH₂PO₄; 8 mM Na₂HPO₄; pH 7A), and subsequently incubated with a mixture of 2 rabbit polyclonal anti-His antibodies (G-18 and H-15, 0.2 μg/ml each; Santa Cruz) at 4° C. overnight. After a further 1 hour incubation at room temperature, the membrane was washed with PBS containing 0.1% Tween-20 (3×10 min), and then exposed to a secondary Horse-Radish Peroxidase (HRP)-conjugated anti-rabbit antibody (DAKO) at room temperature for 2 hours. After washing in PBS containing 0.1% Tween −20 (3×10 minutes), the ECL kit (Amersham Pharmacia) was used to detect the antibodies immobilized onto the membrane, comparing the film with the image of the Coomassie stained gel.

By making use of the above described protocol of protein expression and purification, the presence of sequences allowing secretion into the protein sequences encoded from the cloned ORFs was demonstrated for pIFNFH27, pIFNFH39, and pIFNFH42, which were efficiently purified from the culture medium of the transfected mammalian cells as His-tagged proteins.

### Example 4

### Cell- and Animal-Based Assay for the Validation and Characterization of pIFNFHs

Several assays have been developed for testing specificity, potency, and efficacy of IFNgamma using cell cultures or animal models, as extensively reviewed (Bach E A et al., 1997; Boehm U et al., 1997). Other examples of literature providing examples of human IFNgamma activities are the patent applications disclosing IFNgamma variants (WO 02/81507) or the several therapeutic activities of IFNgamma, alone or in combination with other compounds (WO 95/22328, WO 01/34180, WO 90/03189, EP607258, EP696639, EP490250, EP502997). This prior art provides reliable guidance on how to identify any human IFNgamma activity of the polypeptides of the invention.

Many assays and technologies for generating useful tools and products (antibodies, transgenic animals, radiolabeled proteins, etc.) have been also described in connection to human IFNgamma and/or its receptor (Tura B J et al., 2001; Annicchiarico-Petruzzelli M et al., 2001, Pouly S et al., 2000; Luttmann W et al., 2000; Arai C et al., 1999; Dow S W et al., 1999; Akbar S et al., 1999; Popko B and Baerwald K D, 1999; Zantl N et al., 1998; Sethi S K et al., 1997; Young H A, 1997; Rottenberg M E et al., 2002; Shtrichman R and Samuel C E, 2001; Arai C et al., 1999; Ziesche R et al., 1999; WO 01/34180; EP203580; WO 90/03189; U.S. Pat. No. 5,170,591; WO 02/102312; U.S. Pat. No. 5,666,312; WO 91/07984; U.S. Pat. No. 5,198,212; Docke W D et al., 1997; EP1265996; U.S. Pat. No. 6,036,956; EP 1140990; WO 98/28001; WO 94/12531; WO 94/14497; WO 02/98460; WO 99/09055; WO 00/32634). They can be used to verify the expression and the mechanisms of action of the polypeptides of the invention described under the consensus sequence pIFNFHcon and the related reagents, in connection with possible therapeutic or diagnostic methods and uses.

For example, pIFNFH32 and pIFNFH42, expressed as His-tagged proteins as described above, have a toxic, pro-apoptotic effect on a human leukemic cell line (Jurkat cells) in a system including Fas Ligand and anti-His tag antibody. Apoptosis is quantified by release of LDH (Lactate Dehydrogenase, a cytoplasmic enzyme released in the culture medium when cells are dying) and, after 24 hours of incubation, such effect is comparable observed with IFNgamma, which is known to induce Fas Ligand-mediated apoptosis (Annicchiarico-Petruzzelli M et al., 2001; Li J H et al., 2002).

TABLE I

| Amino Acid | Synonymous Groups |
|---|---|
| Ser | Thr, Ser |
| Arg | Arg, Lys, His |
| Leu | Ile, Val, Leu, Met |
| Pro | Pro |
| Thr | Thr, Ser |
| Ala | Gly, Ala |
| Val | Met, Ile, Val, Leu |
| Gly | Gly, Ala |
| Ile | Ile, Val, Leu, Met |
| Phe | Tyr, Phe |
| Tyr | Phe, Tyr |
| Cys | Cys |
| His | Arg, Lys, His |
| Gln | Asn, Gln |
| Asn | Asn, Gln |
| Lys | Arg, Lys, His |
| Asp | Asp, Glu |
| Glu | Asp, Glu |
| Met | Ile, Val, Leu, Met |
| Trp | Trp |

TABLE II

| Amino Acid | Synonymous Groups |
|---|---|
| Ser | D-Ser, Thr, D-Thr, allo-Thr, Met, D-Met, Met(O), |
| | D-Met(O), L-Cys, D-Cys |
| Arg | D-Arg, Lys, D-Lys, homo-Arg, D-homo-Arg, Met, Ile, |
| | D-.Met, D-Ile, Orn, D-Orn |
| Leu | D-Leu, Val, D-Val, AdaA, AdaG, Leu, D-Leu, Met, D-Met |
| Pro | D-Pro, L-l-thioazolidine-4-carboxylic acid, D-or |
| | L-1-oxazolidine-4-carboxylic acid |
| Thr | D-Thr, Ser, D-Ser, allo-Thr, Met, D-Met, Met(O), |
| | D-Met(O), Val, D-Val |
| Ala | D-Ala, Gly, Aib, B-Ala, Acp, L-Cys, D-Cys |
| Val | D-Val, Leu, D-Leu, Ile, D-Ile, Met, D-Met, AdaA, AdaG |
| Gly | Ala, D-Ala, Pro, D-Pro, Aib, .beta.-Ala, Acp |
| Ile | D-Ile, Val, D-Val, AdaA, AdaG, Leu, D-Leu, Met, D-Met |
| Phe | D-Phe, Tyr, D-Thr, L-Dopa, His, D-His, Trp, D-Trp, |
| | Trans-3,4, or 5-phenylproline, AdaA, AdaG, cis-3,4, |
| | or 5-phenylproline, Bpa, D-Bpa |
| Tyr | D-Tyr, Phe, D-Phe, L-Dopa, His, D-His |
| Cys | D-Cys, S-Me-Cys, Met, D-Met, Thr, D-Thr |
| Gln | D-Gln, Asn, D-Asn, Glu, D-Glu, Asp, D-Asp |
| Asn | D-Asn, Asp, D-Asp, Glu, D-Glu, Gln, D-Gln |
| Lys | D-Lys, Arg, D-Arg, homo-Arg, D-homo-Arg, Met, D-Met, |
| | Ile, D-Ile, Orn, D-Orn |
| Asp | D-Asp, D-Asn, Asn, Glu, D-Glu, Gln, D-Gln |
| Glu | D-Glu, D-Asp, Asp, Asn, D-Asn, Gln, D-Gln |
| Met | D-Met, S-Me-Cys, Ile, D-Ile, Leu, D-Leu, Val, D-Val |

TABLE III

| SEQ | | | |
|---|---|---|---|
| ID | | | |
| NO: | NAME | DIRECTION | 5′-3′ SEQUENCE |
| 41 | CL_IFNFH01_5 | Forward | AACATGACCTCACCAAATAAAC |
| 42 | CL_IFNFH01_3 | Reverse | TCATTTTTTTTTATTCCTTTTCTT |
| | | | TTGTC |
| 43 | CL_IFNFH03_5 | Forward | AACATGACATCACCAAATGAG |
| 44 | CL_IFNFH03_3 | Reverse | TTACAGGTGCCTG CCACTGCAC |
| 45 | CL_IFNFH04_5 | Forward | AACATGACCTCACCAAATGAAC |
| 46 | CL_IFNFH04_3 | Reverse | TCAAGAGACTGATGCATTCTTTAG |
| 47 | CL_IFNFH08_5 | Forward | AACATGACCTCACCAAATGAAC |
| 48 | CL_IFNFH08_3 | Reverse | CTAATTCCGATTAATTCTACTATG |
| 49 | CL_IFNFH10_5 | Forward | AACATGACCTCACCAAATGAG |
| 50 | CL_IFNFH10_3 | Reverse | TCATTGTTTTTTGTTGTTTTTGG |
| | | | TC |
| 51 | CL_IFNFH11_5 | Forward | CACATGACCTCAGGAAATGAAG |
| 52 | CL_IFNFH11_3 | Reverse | TTATTGTTTTTTATTCTTTTTCTT |
| | | | TTG |
| 53 | CL_IFNFH12_5 | Forward | AACATGACCTCACCAAATGAAC |
| 54 | CL_IFNFH12_3 | Reverse | TCAATCAGTTCTGCTATTAAAAAA |
| | | | CTC |
| 55 | CL_IFNFH13_5 | Forward | AACATGACCTCACCAAATGAAC |
| 56 | CL_IFNFH13_3 | Reverse | TTAGGTGTGCTTCATTCTTTTATA |
| | | | TTTTTT |
| 57 | CL_IFNFH14_5 | Forward | AACATGACATCAACAAAGGAAC |
| 58 | CL_IFNFH14_3 | Reverse | TTATATTCTTTTTTCTCTTCTGAC |
| | | | TG |
| 59 | CL_IFNFH15_5 | Forward | AATATGACCTCACCAAATGAAC |
| 60 | CL_IFNFH15_3 | Reverse | CTATTTAAGGCCAATAACTTTTAG |
| 61 | CL_IFNFH20_5 | Forward | AACATGCCCTTACCAAATGAGC |
| 62 | CL_IFNFH20_3 | Reverse | CTATGATGCATTCTTCATTATAC |
| 63 | CL_IFNFH23_5 | Forward | AACATGACCTCACCAAATGAAC |
| 64 | CL_IFNFH23_3 | Reverse | CTATATACTTTCAAATAGCCTGTC |
| 65 | CL_IFNFH27_5 | Forward | AACATGACCTCGCCTAATGAAC |
| 66 | CL_IFNFH27_3 | Reverse | TTAGTTTGCTTCCTCTGACTG |
| 67 | CL_IFNFH31_5 | Forward | AATATGACCTACCAAATGAAC |
| 68 | CL_IFNFH31_3 | Reverse | CTAATACATGCTTCTTTTTTTGTT |
| | | | TG |
| 69 | CL_IFNFH32_5 | Forward | AACATGACCTCACCAAATAAAC |
| 70 | CL_IFNFH32_3 | Reverse | TCAGTATGCCAGTTGATTTTTCA |
| | | | GC |
| 71 | CL_IFNFH36_5 | Forward | AACATGACCTCACCAAACAAAC |
| 72 | CL_IFNFH36_3 | Reverse | TTATTCTGCTTGCTCAATTCTGC |
| 73 | CL_IFNFH37_5 | Forward | AACATGACCTCACTAAATGAAC |
| 74 | CL_IFNFH37_3 | Reverse | CTAATTCTTTTTTTCTGCTCCATA |
| | | | AATTC |
| 75 | CL_IFNFH39_5 | Forward | TCAATGGCCAGACA CCTACAAAC |
| 76 | CL_IFNFH39_3 | Reverse | TCATTCTTCTACTTGATTAATTCT |
| | | | AC |
| 77 | CL_IFNFH42_5 | Forward | TCAATGCCAAGACCAAAGAAC |
| 78 | CL_IFNFH42_3 | Reverse | CTAATTCTTCTTTTCTACTCGAT |
| | | | CC |

TABLE IV

| | | | |
|---|---|---|---|
| SEQ | | | |
| ID | | | |
| NO: | NAME | DIRECTION | 5′-3′ SEQUENCE |

| | | | |
|---|---|---|---|
| 79 | EX1_IFNFH01_5 | Forward | AAGCAGGCTTCGCCA CCAACATG |
| | | | ACCTCACCAAATAAAC |
| 80 | EX1_IFNFH01_3 | Reverse | GTGATGGTGATGGTG TTTTTTTT |
| | | | TATTCCTTTTCTTTTGTC |
| 81 | EX1_IFNFH03_5 | Forward | AAGCAGGCTTCGCCACCAACATGA |
| | | | CATCACCAAATGAG |
| 82 | EX1_IFNFH03_3 | Reverse | GTGATGGTGATGGTG CAGGTGCC |
| | | | TGCCACTGCAC |
| 83 | EX1_IFNFH04_5 | Forward | AAGCAGGCTTCGCCACCATGACCT |
| | | | CACCAAATGAAC |
| 84 | EX1_IFNFH04_3 | Reverse | GTGATGGTGATGGTGAGAGACTGA |
| | | | TGCATTCTTTAG |
| 85 | EX1_IFNFH08_5 | Forward | AAGCAGGCTTCGCCACCAACATGA |
| | | | CCTCACCAAATGAAC |
| 86 | EX1_IFNFH08_3 | Reverse | GTGATGGTGATGGTG ATTCCGAT |
| | | | TAATTCTACTATG |
| 87 | EX1_IFNFH10_5 | Forward | AAGCAGGCTTCG CCACCAACATG |
| | | | ACCTCACCAAATGAG |
| 88 | EX1_IFNFH10_3 | Reverse | GTGATGGTGATGGTG TTGTTTTT |
| | | | TGTTGTTTTTGGTC |
| 89 | EX1_IFNFH11_5 | Forward | AAGCAGGCTTCGCCACCCACATGA |
| | | | CCTCAGGAAATGAAG |
| 90 | EX1_IFNFH11_3 | Reverse | GTGATGGTGATGGTG TTGTTTTT |
| | | | TATTCTTTTTCTTTTG |
| 91 | EX1_IFNFH12_5 | Forward | AAGCAGGCTTCGCCACCAACATGA |
| | | | CCTCACCAAATGAAC |
| 92 | EX1_IFNFH12_3 | Reverse | GTGATGGTGATGGTGATCAGTTCT |
| | | | GCTATTAAAAAACTC |
| 93 | EX1_IFNFH13_5 | Forward | AAGCAGGCTTCGCCACCAACATGA |
| | | | CCTCACCAAATGAAC |
| 94 | EX1_IFNFH13_3 | Reverse | GTGATGGTGATGGTG GGTGTGCT |
| | | | TCATTCTTTTATATTTTTT |
| 95 | EX1_IFNFH14_5 | Forward | AAGCAGGCTTCGCCACCAACATGA |
| | | | CATCAACAAAGGAAC |
| 96 | EX1_IFNFH14_3 | Reverse | GTGATGGTGATGGTG TATTCTTT |
| | | | TTTCTCTTCTGACTG |
| 97 | EX1_IFNFH15_5 | Forward | AAGCAGGCTTCGCCACCAATATGA |
| | | | CCTCACCAAATGAAC |
| 98 | EX1_IFNFH15_3 | Reverse | GTGATGGTGATGGTGTTTAAGGCC |
| | | | AATAACTTTTAG |
| 99 | EX1_IFHFH20_5 | Forward | AAGCAGGCTTCGCCACCAACATGC |
| | | | CCTTACCAAATGAGC |
| 100 | EX1_IFNFH20_3 | Reverse | GTGATGGTGATGGTGTGATGCATT |
| | | | CTTCATTATAC |
| 101 | EX1_IFNFH23_5 | Forward | AAGCAGGCTTCGCCACCAACATGA |
| | | | CCTCACCAAATGAAC |
| 102 | EX1_IFNFH23_3 | Reverse | GTGATGGTGATGGTG TATACTTT |
| | | | CAAATAGCCTGTC |
| 103 | EX1_IFNFH27_5 | Forward | AAGCAGGCTTCGCCACCAACATGA |
| | | | CCTCGCCTAATGAAC |
| 104 | EX1_IFNFH27_3 | Reverse | GTGATGGTGATGGTG GTTTGCTT |
| | | | CCTCTGACTG |
| 105 | EX1_IFNFH31_5 | Forward | AAGCAGGCTTCGCCACCAATATGA |
| | | | CCTCACCAAATGAAC |
| 106 | EX1_IFNFH31_3 | Reverse | GTGATGGTGATGGTG ATACATGC |
| | | | TTCTTTTTTTGTTTG |
| 107 | EX1_IFNFH32_5 | Forward | AAGCAGGCTTCGCCACCAACATGA |
| | | | CCTCACCAAATAAAC |
| 108 | EX1_IFNFH32_3 | Reverse | GTGATGGTGATGGTGGTATGCCAG |
| | | | TTGATTTTTCAGC |
| 109 | EX1_IFNFH36_5 | Forward | AAGCAGGCTTCGCCACCAACATGA |
| | | | CCTCACCAAACAAAC |
| 110 | EX1_IFNFH36_3 | Reverse | GTGATGGTGATGGTGTTCTGCTTG |
| | | | CTCAATTCTGC |
| 111 | EX1_IFNFH37_5 | Forward | AAGCAGGCTTCGCCACCAACATGA |
| | | | CCTCACTAAATGAAC |
| 112 | EX1_IFNFH37_3 | Reverse | GTGATGGTGATGGTGATTCTTTTT |
| | | | TTCTGCTCCATAAATTC |
| 113 | EX1_IFNFH39_5 | Forward | AAGCAGGCTTCGCCACCTCAATGG |
| | | | CCAGACACCTACAAAC |
| 114 | EX1_IFNFH39_3 | Reverse | GTGATGGTGATGGTG TTCTTCTA |
| | | | CTTGA TTAATTCTAC |
| 115 | EX1_IFNFH42_5 | Forward | AAGCAGGCTTCGCCACCTCAATGC |
| | | | CAAGACACCAAAGAAC |
| 116 | EX1_IFNFH42_3 | Reverse | GTGATGGTGATGGTGATTCTTCTT |
| | | | TTCTACTCGATCC |

TABLE V

| SEQ | | | |
|---|---|---|---|
| ID | | | |
| NO: | NAME | DIRECTION | 5′-3′ SEQUENCE |
| 117 | EX2_IFNFH01_5 | Forward | GGGGACAAGTTTGTACAAAAAAGC |
| | | | AGGCTTCGC CACCAACATGACCT |
| | | | CACCAAATAAAC |
| 118 | EX2_IFNFH01_3 | Reverse | GGGGACCACTTGTACA AGAAAGC |
| | | | TGGGTTTCAATGGTGATGGTGATG |
| | | | GTGTTTTTTTTTATTCCTTTTCTT |
| | | | TTGTC |
| 119 | EX2_IFNFH03_5 | Forward | GGGGACAAGTTTGTACAAAAAAGC |
| | | | AGGCTTCGCCACCAACATGACATC |
| | | | ACCAAATGAG |
| 120 | EX2_IFNFH03_3 | Reverse | GGGGACCACTT TGTACAAGAAAG |
| | | | CTGGGTTTCAATGGTGATGGTGAT |
| | | | GGTGCAGGTGCCTGCCACTGCAC |
| 121 | EX2_IFNFH04_5 | Forward | GGGGACAAGTTTGTACAAAAAAGC |
| | | | AGGCTTCGCCACCAACATGACCTC |
| | | | ACCAAATGAAC |
| 122 | EX2_IFNFH04_3 | Reverse | GGGGACCACTTTGTACA AGAAAG |
| | | | CTGGGTTTCAATGGTGATGGTGAT |
| | | | GGTGAGAGACTGATGCATTCTTTA |
| | | | G |
| 123 | EX2_IFNFH08_5 | Forward | GGGGACAAGTTTGTACAAAAAAGC |
| | | | AGGCTTCGCCACCAACATGACCTC |
| | | | ACCAAATGAAC |
| 124 | EX2_IFNFH08_3 | Reverse | GGGGACCACTTTGTACA AGAAAG |
| | | | CTGGGTTTCAATGGTGATGGTGAT |
| | | | GGTGATTCCGATTAATTCTACTAT |
| | | | G |
| 125 | EX2_IFNFH10_5 | Forward | GGGGACAAGTTTGTACAAAAAAGC |
| | | | AGGCTTCGCCACCAACATGACCTC |
| | | | ACC AAATGAG |
| 126 | EX2_IFNFH10_3 | Reverse | GGGGACCACTTTGTACAAGAAAGC |
| | | | TGGGTTTCAATGGTGATGGTGATG |
| | | | GTGTTGTTTTTTGTTGTTTTTGGT |
| | | | C |
| 127 | EX2_IFNFH11_5 | Forward | GGGGACAAGTTTGTACAAAAAAGC |
| | | | AGGCTTCGCCACCCACATGACCTC |
| | | | AGGAAATGAAG |
| 128 | EX2_IFNFH11_3 | Reverse | GGGGACCACTTTGTACAAGAAAGC |
| | | | TGGGTTTCA ATGGTGATGGTGAT |
| | | | GGTG TTGTTTTTTATTCTTTTTC |
| | | | TTTTG |
| 129 | EX2_IFNFH12_5 | Forward | GGGGACAAGTTTGTACAAAAAAGC |
| | | | AGGCTTCGCCACCAACATGACCTC |
| | | | ACCAAATGAAC |
| 130 | EX2_IFNFH12_3 | Reverse | GGGGACCACTTTGTACA AGAAAG |
| | | | CTGGGTTTCAATGGTGATGGTGAT |
| | | | GGTGATCAGTTCTGCTATTAAAAA |
| | | | ACTC |
| 131 | EX2_IFNFH13_5 | Forward | GGGGACAAGTTTGTACAAAAAAGC |
| | | | AGGCTTCGCCACCAACATGACCTC |
| | | | ACCAAATGAAC |
| 132 | EX2_IFNFH13_3 | Reverse | GGGGACCACTTTGTACA AGAAAG |
| | | | CTGGGTTTCAATGGTGATGGTGAT |
| | | | GGTGGGTGTGCTTCATTCTTTTAT |
| | | | ATTTTTT |
| 133 | EX2_IFNFH14_5 | Forward | GGGGACAAGTTTGTACAAAAAAGC |
| | | | AGGCTTCGCCACCAACATGACATC |
| | | | AACAAAGGAAC |
| 134 | EX2_IFNFH14_3 | Reverse | GGGGACCATTTGTACAAGAAAGCT |
| | | | GGGTTTCAATGGTGATGGTGATGG |
| | | | TG TATTCTTTTTTCTCTTCTGAC |
| | | | TG |
| 135 | EX2_IFNFH15_5 | Forward | GGGGACAAGTTTGTACAAAAAAGC |
| | | | AGGCTTCGCCACCAATATGACCTC |
| | | | ACCAAATGAAC |
| 136 | EX2_IFNFH15_3 | Reverse | GGGGACCACTTTGTACA AGAAAG |
| | | | CTGGGTTTCAATGGTG ATGGTGA |
| | | | TGGTGTTTAAGGCCAATAACTTTT |
| | | | AG |
| 137 | EX2_IFNFH20_5 | Forward | GGGGACAAGTTTGTACAAAAAAGC |
| | | | AGGCTTCGCCACCAACATGCCCTT |
| | | | ACCAAATGAGC |
| 138 | EX2_IFNFH20_3 | Reverse | GGGGACCACTTTGTACA AGAAAG |
| | | | CTGGGTTTCAATGGTGATGGTGAT |
| | | | GGTGTGATGCATTCTTCATTATAC |
| 139 | EX2_IFNFH23_5 | Forward | GGGGACAAGTTTGTACAAAAAAGC |
| | | | AGGCTTCGCCACCAACATGACCTC |
| | | | ACCAAATGAAC |
| 140 | EX2_IFNFH23_3 | Reverse | GGGGACCACTTTGTACA AGAAAG |
| | | | CTGGGTTTCAATGGTGATGGTGAT |
| | | | GGTGTATACTTTCAAATAGCCTGT |
| | | | C |
| 141 | EX2_IFNFH27_5 | Forward | GGGGACAAGTTTGTACAAAAAAGC |
| | | | AGGCTTCGCCACCAACATGACCTC |
| | | | GCCTAATGAAC |
| 142 | EX2_IFNFH27_3 | Reverse | GGGGACCACTTTGTACA AGAAAG |
| | | | CTGGGTTTCAATGGTGATGGTGAT |
| | | | GGTGGTTTGCTTCCTCTGACTG |
| 143 | EX2_IFNFH31_5 | Forward | GGGGACAAGTTTGTACAAAAAAGC |
| | | | AGGCTTCGCCACCAATATGACCTC |
| | | | ACCAAATGAAC |
| 144 | EX2_IFNFH31_3 | Reverse | GGGGACCACTTTGTACA AGAAAG |
| | | | CTGGGTTTCAATGGTGATGGTGAT |
| | | | GGTGATACATGCTTCT TTTTTTG |
| | | | TTTG |
| 145 | EX2_IFNFH32_5 | Forward | GGGGACAAGTTTGTACAAAAAAGC |
| | | | AGGCTTCGCCACCAACATGACCTC |
| | | | ACCAAATAAAC |
| 146 | EX2_IFNFH32_3 | Reverse | GGGGACCACTTTGTACA AGAAAG |
| | | | CTGGGTTTCAATGGTGATGGTGAT |
| | | | GGTGGTATGCCAGTTGATTTTTC |
| | | | AGC |
| 147 | EX2_IFNFH36_5 | Forward | GGGGACAAGTTTGTACAAAAAAGC |
| | | | AGGCTTCGCCACCAACATGACCTC |
| | | | ACCAAACAAAC |
| 148 | EX2_IFNFH36_3 | Reverse | GGGGACCACTTTGTACA AGAAAG |
| | | | CTGGGTTTCAATGGTGATGGTGAT |
| | | | GGTGTTCTGCTTGCTCAATTCTGC |
| 149 | EX2_IFNFH37_5 | Forward | GGGGACAAGTTTGTACAAAAAAGC |
| | | | AGGCTTCGCCACCAA CATGACCT |
| | | | CACTAAATGAAC |
| 150 | EX2_IFNFH37_3 | Reverse | GGGGACCACTTTGTACA AGAAAG |
| | | | CTGGGTTTCAATGGTGATGGTGAT |
| | | | GGTGATTCTTTTTTTCTGCTCCAT |
| | | | AAATTC |
| 151 | EX2_IFNFH39_5 | Forward | GGGGACAAGTTTGTACAAAAAAGC |
| | | | AGGCTTCGCCACCTCAATGGCCAG |
| | | | ACACCTACAAAC |
| 152 | EX2_IFNFH39_3 | Reverse | GGGGACCACTTTGTA CAAGAAAG |
| | | | CTGGGTTTCAATGGTGATGGTGAT |
| | | | GGTGTTCTTCTACTTGATTAATTC |
| | | | TAC |
| 153 | EX2_IFNFH42_5 | Forward | GGGGACAAGTTTGTACAAAAAAGC |
| | | | AGGCTTCGCCACCTCAATGCCAAG |
| | | | ACACCAAAGAAC |
| 154 | EX2_IFNFH42_3 | Reverse | GGGGACCACTTTGTACA AGAAAG |
| | | | CTGGGTTTCAATGGTGATGGTGAT |
| | | | GGTGATTCTTCTTTTCTACTCGAT |
| | | | CC |

### REFERENCES

Altschul S F et al., J Mol Biol, 215: 403-10.1990.
Akbar S et al., Biochem Biophys Res Commun, 259: 294-9, 1999.
Andersen D C and Krummen L, Curr Opin Biotechnol, 13: 117-23, 2002.
Annicchlarico-Petrulli M et al., Med Pediatr Oncol, 36: 115-7, 2001.
Arai C et al., In Vivo, 13:445-51, 1999.
Bach E A et al., Annu Rev Immunol, 15:563-91, 1997.
Baker K N et al., Trends Biotechnol, 20: 149-56, 2002.
Blagoev B and Pandey A, Trends Biochem Sci, 26: 639-41, 2001.
Bock A, Science, 292: 453-4, 2001.
Boehm U et al., Annu Rev Immunol, 15:749-795, 1997.
Brown A et al., J Pept Sci, 2: 40-46, 1996.
Bunz F, Curr Opin Oncol, 14: 73-8, 2002.
Burgess R R and Thompson N E, Curr Opin Biotechnol, 12: 450-4, 2001.
Chambers S P, Drug Disc Today, 14: 759-765, 2002.
Choy E H and Panayi G S, N Engl J Med, 344: 907-16, 2001.
Chu L and Robinson D K, Curr Opin Biotechnol, 13: 304-8, 2001.
Cleland J L et al., Curr Opin Biotechnol, 12: 212-9, 2001.
Coleman T A et al., Gene 190:163-171, 1997.
Constans A, The Scientist, 16: 37, 2002.
Davis B G and Robinson M A, Curr Opin Drug Discov Devel, 5: 279-88, 2002.
Docke W D et al., Nat Med, 3:678-681, 1997.
Dow S W et al., Hum Gene Ther, 10: 1905-14, 1999.
Dower S K, Nat Immunol, 1: 367-8, 2000.
Dougherty D A, Curr Opin Chem Biol, 4: 645-52, 2000.
Garnett M C, Adv Drug Deliv Rev, 53: 171-216, 2001.
Gavilondo J V and Larrick J W, Biotechniques, 29: 128-136, 2000.
Gendel S M, Ann N Y Acad Sci, 964: 87-98, 2002.
Giddings G, Curr Opin Blotechnol, 12: 450-4, 2001.
Gish W and States D J, Nat Genet, :266-72, 1993.
Golebiowski A et al., Curr Opin Drug Discov Devel, 4: 428-34, 2001.
Gupta P et al., Drug Discov Today, 7: 569-579, 2002.
Haupt K, Nat Biotechnol, 20: 884-5, 2002.
Hruby V J and Balse P M, Curr Med Chem, 7: 945-70, 2000.
Johnson D E and Wolfgang G H, Drug Discov Today, 5: 445-454, 2000.
Kane J F, Curr Opin Blotechnol, 6: 494-500, 1995.
Kolb A F, Cloning Stem Cells, 4: 65-80, 2002.
Kurowa Y et al., Nat Blotechnol, 20: 889-94, 2002.
Lewis D L, Nat Genet, 32: 107-8, 2002.
Li J H et al., Am J Pathol, 161: 1485-95, 2002.
Lin Cereghino G P et al., Curr Opin Biotechnol, 13: 329-332, 2001.
Lowe C R et al., J Biochem Biophys Methods, 49: 561-74, 2001.
Luo B and Prestwich G D, Exp Opin Ther Patents, 11: 1395-1410, 2001.
Luttmann W et al., Scand J Immunol, 51: 54-9, 2000.
Martoglio B and Dobberstein B, Trends Cell Biol, 8: 410-5, 1998.
Michiels L et al., Int J Biochem Cell Biol, 30: 505-16, 1998.
Mulder N J and Apweiler R. Genome Biol, 3(1):REVIEWS2001, 2002
Nilsson J et al., Protein Expr Purif, 11: 1-16, 1997.
Paddison P J, Proc Natb Acad Sci USA, 99: 1443-8, 2002.
Pearson W R and Miller W, Methods Enzymol, 210: 575-601, 1992.
Pellois J P et al., Nat Biotechnol, 20: 922-6, 2002.
Pillal O and Panchagnula R. Cur Opin Chem Biol, 5: 447-451, 2001.
Popko B and Baerwald K D, Neurochem Res, 24: 331-8, 1999.
Pouly S et al., J Neuropathol Exp Neurol, 59: 280-92, 2000.
Rehm B H, Appl Microbiol Biotechnol, 57: 579-92, 2001.
Robinson C R, Nat Biotechnol, 20: 879-880, 2002.
Rottenberg M E et al., Curr Opin Immunol, 14: 444-51, 2002.
Schellekens H, Nat Rev Drug Discov, 1: 457-62, 2002.
Sethi S K et al., Clin Exp Immunol, 110: 362-9, 1997.
Shaw A C et al., Electrophoresis, 20: 984-93, 1999.
Sheibani N. Prep Biochem Biotechnol, 29: 77-90, 1999.
Stein C A, J Clin Invest, 108: 641-4, 2001.
Stein L D, Methods Biochem Anal, 43: 413-49, 2001.
Shtrichman R and Samuel C E, Curr Opin Microbiol, 4: 251-9, 2001.
Templin M F et al., Trends Biotechnol, 20: 160-6, 2002.
Tribbick G, J Immunol Methods, 267: 27-35, 2002.
Tura B J et al., Int J Exp Pathol, 82: 317-26, 2001.
van den Burg B and Eijsink V, Curr Opin Biotechnol, 13: 333-337, 2002.
van Dijk M A and van de Winkel J G, Curr Opin Chem Biol, 5: 368-74, 2001.
Vandenbroeck K and Gods A, Trends Pharmacol Sci, 24: 284-9, 2003.
Villain M et al., Chem Biol, 8: 673-9, 2001.
Younes H M and Amsden B G, J Pharm Sci, 91:2-17, 2002.
Young H A, Methods, 11: 112-5, 1997.
Zantl N et al., Infect Immun, 66: 2300-9, 1998.
Ziesche R et al., N Engl J Med, 341:1264-9, 1999.

1-49. (canceled)

50. A composition of matter comprising:
a) an isolated polypeptide presenting at least one activity of human IFNgamma, and comprising a sequence having at least 80% of homology with the complete sequence of pIFNFHcon (SEQ ID NO: 156) and no more than nine non-conservative mutations in the positions corresponding to Ala10, Gly12, Arg26, Ala31, Lys35, Phe47, Gln55, Glu57, Lys63, and Ile75 in pIFNFHcon;
b) an isolated polypeptide that comprises a sequence having at least 80% of homology with the complete sequence of pIFNFHcon and no non-conservative mutations in the positions corresponding to Ala10, Gly12, Arg26, Ala31, Lys35, Phe47, Gln55, Glu57, Lys63, and Ile75 in pIFNFHcon;
c) an isolated sequence chosen from pIFNFH15 (SEQ ID NO: 20), pIFNFH32 (SEQ ID NO: 32), and pIFNFH37 (SEQ ID NO: 36);
d) an isolated polypeptide that comprises a sequence having at least 80% of homology with the complete sequence of pIFNFHcon and one or two non-conservative mutations in the positions corresponding to Ala10, Gly12, Arg26, Ala31, Lys35, Phe47, Gln55, Glu57, Lys63, and Ile75 in pIFNFHcon;
e) an isolated polypeptide as set forth in d) that comprises a sequence chosen from pIFNFH04 (SEQ ID NO: 6), pIFNFH03 (SEQ ID NO: 4), pIFNFH08 (SEQ ID NO: 8), pIFNFH20 (SEQ ID NO: 22), pIFNFH23 (SEQ ID NO: 24), pIFNFH12 (SEQ ID NO: 14), pIFNFH25 (SEQ ID NO: 26), pIFNFH13 (SEQ ID NO: 16), pIFNFH14 (SEQ ID NO: 18), pIFNFH36 (SEQ ID NO: 34), and pIFNFH39 (SEQ ID NO: 38);
f) an isolated polypeptide that comprises a sequence having at least 80% of homology with the complete sequence of pIFNFHcon and three, four, or five non-conservative mutations in the positions corresponding to Ala10, Gly12, Arg26, Ala31, Lys35, Phe47, Gln55, Glu57, Lys63, and Ile75 in pIFNFHcon;
g) an isolated polypeptide as set forth in f) that comprises a sequence chosen from pIFNFH11 (SEQ ID NO: 12), pIFNFH27 (SEQ ID NO: 28), pIFNFH01 (SEQ ID NO: 2), pIFNFH31 (SEQ ID NO: 30), pIFNFH10 (SEQ ID NO: 10), and pIFNFH42 (SEQ ID NO: 40);
h) an isolated polypeptide that is a variant, a mature form, or an active fragment of the amino acid sequences SEQ ID NO: 2, 4, 6, 8, 10, 12, 14, 16, 18, 20, 22, 24, 26, 28, 30, 32, 34, 36, 38, and 40;
i) an isolated polypeptide that is a naturally occurring allelic variant of the sequences SEQ ID NO: 2, 4, 6, 8, 10, 12, 14, 16, 18, 20, 22, 24, 26, 28, 30, 32, 34, 36, 38, and 40;
j) an isolated polypeptide as set forth in i), wherein the variant is the translation of one or more single nucleotide polymorphisms;
k) a fusion protein comprising a polypeptide according any one of (a) through (j) and a sequence heterologous to pIFNFHcon;
l) a ligand binding specifically to a polypeptide according to any one of (a) through (j);
m) a polypeptide as set forth in any one of a) through l), wherein said polypeptides are in the form of active fractions, precursors, salts, or derivatives;
n) a polypeptide as set forth in (a) through (l), wherein said polypeptides are in the form of active conjugates or complexes with a molecule chosen from radioactive labels, fluorescent labels, biotin, or cytotoxic agents;
o) a peptide mimetic designed on the sequence and/or the structure of a polypeptide as set forth in (a);
p) an isolated nucleic acid encoding for an isolated polypeptide as set forth in any one of (a) through (k);
q) an isolated nucleic acid comprising the coding portion of a sequence selected from the group consisting of SEQ ID NOs: 1, 3, 5, 7, 9, 11, 13, 15, 17, 19, 21, 23, 25, 27, 29, 31, 33, 35, 37, and 39, or the complement of said sequence;
r) a purified nucleic acid which hybridizes under high stringency conditions with a nucleic acid selected from the group consisting of SEQ ID NOs: 1, 3, 5, 7, 9, 11, 13, 15, 17, 19, 21, 23, 25, 27, 29, 31, 33, 35, 37, and 39, or a complement of said nucleic acid;
s) a vector comprising a nucleic acid encoding for an isolated polypeptide as set forth in any one of (a) through (k); comprising the coding portion of a sequence selected from the group consisting of SEQ ID NOs: 1, 3, 5, 7, 9, 11, 13, 15, 17, 19, 21, 23, 25, 27, 29, 31, 33, 35, 37, and 39, or the complement of said sequence; or comprising a nucleic acid which hybridizes under high stringency conditions with a nucleic acid selected from the group consisting of SEQ ID NOs: 1, 3, 5, 7, 9, 11, 13, 15, 17, 19, 21, 23, 25, 27, 29, 31, 33, 35, 37, and 39, or a complement of said nucleic acid;
t) a vector comprising a nucleic acid encoding for an isolated polypeptide as set forth in any one of (a) through (k); comprising the coding portion of a sequence selected from the group consisting of SEQ ID NOs: 1, 3, 5, 7, 9, 11, 13, 15, 17, 19, 21, 23, 25, 27, 29, 31, 33, 35, 37, and 39, or the complement of said sequence; or comprising a nucleic acid which hybridizes under high stringency conditions with a nucleic acid selected from the group consisting of SEQ ID NOs: 1, 3, 5, 7, 9, 11, 13, 15, 17, 19, 21, 23, 25, 27, 29, 31, 33, 35, 37, and 39, or a complement of said nucleic acid;
u) a vector comprising a nucleic acid encoding for an isolated polypeptide as set forth in any one of (a) through (k); comprising the coding portion of a sequence selected from the group consisting of SEQ ID NOs: 1, 3, 5, 7, 9, 11, 13, 15, 17, 19, 21, 23, 25, 27, 29, 31, 33, 35, 37, and 39, or the complement of said sequence; or comprising a nucleic acid which hybridizes under high stringency conditions with a nucleic acid selected from the group consisting of SEQ ID NOs: 1, 3, 5, 7, 9, 11, 13, 15, 17, 19, 21, 23, 25, 27, 29, 31, 33, 35, 37, and 39, or a complement of said nucleic acid, wherein said nucleic acid molecule is operatively linked to expression control sequences allowing expression in prokaryotic or eukaryotic host cells of the encoded polypeptide;
v) a host cell transformed with a vector or a nucleic acid according to any one of (p), (q), (r), (s), (t), or (u);
w) a transgenic animal cell that has been transformed with a vector or a nucleic acid according to any one of (p), (q), (r), (s), (t), or (u) and having enhanced or reduced expression levels of a polypeptide;
x) a transgenic non-human organism that has been transformed to have enhanced or reduced expression levels of a polypeptide according to any one of (a) though (j);
y) a compound that enhances the expression level of a polypeptide a polypeptide according to any one of (a) though (j) in a cell or in an animal; or
z) a compound that reduces the expression level of a polypeptide according to any one of (a) though (j) in a cell or in an animal.

51. The composition of matter according to claim 50, wherein said fusion protein comprises one or more amino acid sequence belonging a protein sequences selected from: membrane-bound protein, immunoglobulin constant region, multimerization domains, extracellular proteins, signal peptide-containing proteins, or export signal-containing proteins.

52. The composition of matter according to claim 50, wherein said ligand antagonizes or inhibits the IFNgamma-related activity of said polypeptide.

53. The composition of matter according to claim 52, wherein said ligand is a monoclonal antibody, a polyclonal antibody, a humanized antibody, or an antigen binding fragment thereof.

54. The composition of matter according to claim 52, wherein said ligand corresponds to the extracellular domain of a membrane-bound protein.

55. The composition of matter according to claim 50, wherein said composition of matter further comprises a pharmaceutically acceptable carrier.

56. The composition of matter according to claim 50, wherein said compound that enhances the expression level of a polypeptide a polypeptide according to any one of (a) though (j) is an antisense oligonucleotide or a small interfering RNA

57. A method of using the composition of matter for producing cells capable of expressing a polypeptide; for making a polypeptide; the preparation of pharmaceutical compositions comprising a polypeptide; for the treatment or prevention of diseases needing the increase of a human IFNgamma-related activity; for the treatment or prevention of a disease associated to the excessive human IFNgamma-related activity; for the treatment or prevention of diseases related to a polypeptide; for screening candidate compounds effective to treat a disease related to a polypeptide; method for determining the activity and/or the presence of the polypeptide; or for determining the presence or the amount of a transcript or of a nucleic acid encoding the polypeptide, wherein said polypeptide is:
a) an isolated polypeptide presenting at least one activity of human IFNgamma, and comprising a sequence having at least 80% of homology with the complete sequence of pIFNFHcon (SEQ ID NO: 156) and no more than nine non-conservative mutations in the positions corresponding to Ala10, Gly12, Arg26, Ala31, Lys35, Phe47, Gln55, Glu57, Lys63, and Ile75 in pIFNFHcon;
b) an isolated polypeptide that comprises a sequence having at least 80% of homology with the complete sequence of pIFNFHcon and no non-conservative mutations in the positions corresponding to Ala10, Gly12, Arg26, Ala31, Lys35, Phe47, Gln55, Glu57, Lys63, and Ile75 in pIFNFHcon;
c) an isolated sequence chosen from pIFNFH15 (SEQ ID NO: 20), pIFNFH32 (SEQ ID NO: 32), and pIFNFH37 (SEQ ID NO: 36);
d) an isolated polypeptide that comprises a sequence having at least 80% of homology with the complete sequence of pIFNFHcon and one or two non-conservative mutations in the positions corresponding to Ala10, Gly12, Arg26, Ala31, Lys35, Phe47, Gln55, Glu57, Lys63, and Ile75 in pIFNFHcon;
e) an isolated polypeptide as set forth in d) that comprises a sequence chosen from pIFNFH04 (SEQ ID NO: 6), pIFNFH03 (SEQ ID NO: 4), pIFNFH08 (SEQ ID NO: 8), pIFNFH20 (SEQ ID NO: 22), pIFNFH23 (SEQ ID NO: 24), pIFNFH12 (SEQ ID NO: 14), pIFNFH25 (SEQ ID NO: 26), pIFNFH13 (SEQ ID NO: 16), pIFNFH14 (SEQ ID NO: 18), pIFNFH36 (SEQ ID NO: 34), and pIFNFH39 (SEQ ID NO: 38);
f) an isolated polypeptide that comprises a sequence having at least 80% of homology with the complete sequence of pIFNFHcon and three, four, or five non-conservative mutations in the positions corresponding to Ala10, Gly12, Arg26, Ala31, Lys35, Phe47, Gln55, Glu57, Lys63, and Ile75 in pIFNFHcon;
g) an isolated polypeptide as set forth in f) that comprises a sequence chosen from pIFNFH11 (SEQ ID NO: 12), pIFNFH27 (SEQ ID NO: 28), pIFNFH01 (SEQ ID NO: 2), pIFNFH31 (SEQ ID NO: 30), pIFNFH10 (SEQ ID NO: 10), and pIFNFH42 (SEQ ID NO: 40);
h) an isolated polypeptide that is a variant, a mature form, or an active fragment of the amino acid sequences SEQ ID NOs: 2, 4, 6, 8, 10, 12, 14, 16, 18, 20, 22, 24, 26, 28, 30, 32, 34, 36, 38, and 40;
i) an isolated polypeptide that is a naturally occurring allelic variant of the sequences SEQ ID NOs: 2, 4, 6, 8, 10, 12, 14, 16, 18, 20, 22, 24, 26, 28, 30, 32, 34, 36, 38, and 40;
j) an isolated polypeptide as set forth in i), wherein the variant is the translation of one or more single nucleotide polymorphisms; or
k) a fusion protein comprising a polypeptide according any one of (a) through (j) and a sequence heterologous to pIFNFHcon.

58. The method according to claim 57, wherein said method for producing cells capable of expressing said polypeptide comprises genetically engineering cells with a vector or a nucleic acid encoding said polypeptide.

59. The method according to claim 57, wherein said method for making a polypeptide comprises culturing a cell under conditions in which a nucleic acid or vector encoding said polypeptide is expressed and recovering the polypeptide encoded by said nucleic acid or vector from cell culture.

60. The method according to claim 57, wherein said method of treating or preventing a disease when the increase of a human IFNgamma-related activity of a polypeptide of any of the claims from 1 to 10 is needed comprises the administration of said polypeptide, a peptide mimetic or a compound that increases the activity of human IFNgamma-related polypeptide.

61. The method according to claim 57, wherein said method for the treatment or prevention of diseases needing the increase of a human IFNgamma-related activity of a polypeptide comprises the administration of a therapeutically effective amount of said polypeptide, a peptide mimetic, or a compound that increases the activity of a human IFNgamma-related polypeptide.

62. The method according to claim 57, wherein said method for the treatment or prevention of diseases associated to the excessive human IFNgamma-related activity of a comprises the administration of a composition comprising a therapeutically effective amount of a ligand or of a compound.

63. The method according to claim 57, wherein said method for screening candidate compounds effective to treat a disease related to said polypeptide comprises:
a) contacting a cell or a transgenic non-human organism having enhanced or reduced expression levels of the polypeptide with a candidate compound; and
b) determining the effect of the compound on the animal or on the cell.

64. The method according to claim 57, said method for identifying a candidate compound as an antagonist/inhibitor or agonist/activator of said polypeptide comprises:
(a) contacting said polypeptide and said compound with a mammalian cell or a mammalian cell membrane capable of binding the polypeptide; and
(b) measuring whether the compound blocks or enhances the interaction of the polypeptide, or the response that results from such interaction, with the mammalian cell or the mammalian cell membrane.

65. The method according to claim 57, wherein said method for determining the activity and/or the presence of said polypeptide of in a sample comprises:
(a) providing a protein-containing sample;
(b) contacting said sample with a ligand of; and
(c) determining the presence of said ligand bound to said polypeptide.

66. The method according to claim 57, wherein said method for determining the presence or the amount of a transcript or of a nucleic acid encoding said polypeptide in a sample comprises:
(a) providing a nucleic acids-containing sample;
(b) contacting said sample with a nucleic acid; and
(c) determining the presence or amount of a transcript or of a nucleic acid encoding said polypeptide.

67. The method according to claim 66, wherein said nucleic acid comprises any of the sequences SEQ ID NOs: 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, or 78.

68. The method according to claim 66, wherein said determining comprises polymerase chain reaction, nucleic acid sequencing, or nucleic acid hybridization.

69. A method of secreting a polypeptide comprising transforming a cell with a nucleic acid encoding a polypeptide comprising pIFNFH01 (SEQ ID NO: 2), pIFNFH03 (SEQ ID NO: 4), pIFNFH04 (SEQ ID NO: 6), pIFNFH08 (SEQ ID NO: 8), pIFNFH10 (SEQ ID NO: 10), pIFNFH11 (SEQ ID NO: 12), pIFNFH12 (SEQ ID NO: 14), pIFNFH13 (SEQ ID NO: 16), pIFNFH14 (SEQ ID NO: 18), pIFNFH15 (SEQ ID NO: 20), pIFNFH20 (SEQ ID NO: 22), pIFNFH23 (SEQ ID NO: 24), pIFNFH25 ID NO: 26), pIFNFH27 (SEQ ID NO: 28), pIFNFH31 (SEQ ID NO: 30), pIFNFH32 (SEQ ID NO: 32), pIFNFH36 (SEQ ID NO: 34), pIFNFH37 (SEQ ID NO: 36) pIFNFH39 (SEQ ID NO: 38), or pIFNFH42 (SEQ ID NO: 40) fused to a heterologous polypeptide.

70. The method according to claim 69, wherein said nucleic acid is chosen from pIFNFH27 (SEQ ID NO: 28), pIFNFH39 (SEQ ID NO: 38), and pIFNFH42 (SEQ ID NO: 40), or any secreted fragment thereof.


SEQUENCE LISTING (158 sequences)

SEQ ID NO: 1 - DNA, 360 bp, Homo sapiens
aacatgacctcaccaaataaactaaataagctaccagggaccaaccctggagaaacagaaatatgtgaccttttagatagagaattcaaaatagctgtgttgaggaaactcaaaaaatatcaagatgatacagagaagaagttcagaattctatcagataaatttaacaaagagattgaaatattaaaaaataatcaagcagaaattctggagctgaaaaatttgactggaatactgaagaatgtgccagggtcttttaatagcagaattgatggagcaaaaggaagaattagtaagcctgaagacaggttatttgaaaatacacagaggagacaaaagaaaaggaataaaaaaaaatga

SEQ ID NO: 2 - protein, 118 aa, Homo sapiens [sequence omitted]

SEQ ID NO: 3 - DNA, 414 bp, Homo sapiens
aacatgacatcaccaaatgagttaaatgaggcagcaggaactactcccaaagaaacagagatatgtgacatttcagacagagaattcaaaatagctttgttgaagaaactcaaagaaattcaagataatacggagaaggaactcagaattctatcagataaatttaacaaggagattgaaatgattaaaaagaaccaagcagaaattctggagctaaaaaatgcaggtggcatattgaaaatgcatcagagttggctgggcatggtggctcacgcctgtaatcccagtactttgggaagccgaggtgggtggatcacgagttcaggagttcaagaccagcctggccaaggcagtgaaacctcatctctactaaaaatacaaaaattagctgggtgcagtggcaggcacctgtaa

SEQ ID NO: 4 - protein, 136 aa, Homo sapiens [sequence omitted]

SEQ ID NO: 5 - DNA, 258 bp, Homo sapiens
aacatgacctcaccaaatgaactaaataaggcaccagggaccaatcctggagaaacagagatgtatgacctttcagacagagaattcaaaacagctattttgaggaaactcaaagaaattcaagataacacaaagaaggaattcagaattctatcagataaatttaacaaacagatcgaaataattaaaaagaatcaagcagaaattctagagctgaaaaatgtaattgatatactaaagaatgcatcagtctcttga

SEQ ID NO: 6 - protein, 84 aa, Homo sapiens
MTSPNELNKAPGTNPGETEMYDLSDREFKTAILRKLKEIQDNTKKEFRILSDKFNKQIEIIKKNQAEILELKNVIDILKNASVS

SEQ ID NO: 7 - DNA, 294 bp, Homo sapiens
aacatgacctcaccaaatgaacttagtaaggcaccagggaccaatcagggagaaacagagatatatgacctttcagacacagaattcaaaatagctgttttgagaaactcaaagaagaaactcaaagaaattcaggataacacagagaaggaattcagaattctatcagataaatttaacaaagagattgaaataattaaaaagaatcaagcagaaattctagagttgaaaaatgcaattgacatgctgaataatgcatcagattatcttcatagtagaattaatcggaattag

SEQ ID NO: 8 - protein, 96 aa, Homo sapiens [sequence omitted]

SEQ ID NO: 9 - DNA, 360 bp, Homo sapiens
aacatgacctcaccaaatgaggtaaataaggtaccaatgaccaaccctggagaaacggagatatgtgacctttcagaccaaaaattaaaaatagctgtgatgaggaaactcaaagaaattcaagataacacagagaaagaattcaaaattctatcacgtaaatttaacaaaaagattggattaattgaaaataatcaagcagaaattttggagctgaaaaatgcaattggcatactgaagaatgcatcagagtcctttaatagcaatatgtatcaagcagaagacagaattagtgagcttaaatacaggctatttgaaaatacacagtcagaggagaccaaaaacaacaaaaaacaatga

SEQ ID NO: 10 - protein, 118 aa, Homo sapiens [sequence omitted]

SEQ ID NO: 11 - DNA, 360 bp, Homo sapiens
cacatgacctcaggaaatgaagtaaataaggcaccagggaccaatcttggagaaacagagatatgtgacctttcagatacagaactcagaataactgtgttgaggaaactcaatgaaattaaagataacacagagatggaattcagaattttgtcagataaatttaagaaagagattgaaataattaaaaggaatcaagcagaaattctggagctgaaaaatgcaattggcatactgaagaatgcatcagagtttttaaatagaagaacagatcaagcagcagaaaaatctagtgagcctgaagacagactatttgaaaatacacagaggtctcaaaagaaaaagaataaaaaacaataa

SEQ ID NO: 12 - protein, 118 aa, Homo sapiens [sequence omitted]

SEQ ID NO: 13 - DNA, 276 bp, Homo sapiens
aacatgacctcaccaaatgaactgaataagccaccagggaccaatcctggagaaacagaaatatgtgacctttcagacaaagaattcaaaatagctgtgttgaagaaactcaacgaagctcaagatagcacagagaaggaattcagaattctatcagataaatgtaacaaagacattaaaataattaaaaagaatcaagcagaatttctgaagctgaaagatgcaattggaatactgaaggatgcatcagagttttttaatagcagaactgattga

SEQ ID NO: 14 - protein, 90 aa, Homo sapiens [sequence omitted]

SEQ ID NO: 15 - DNA, 369 bp, Homo sapiens
aacatgacctcaccaaatgaactaaataaggcaccagggaccaatcctggagaaactgagatatgtgacctttcagacagaaaattcaaaagagctgtgttgaagaaactcaaagaaattcaaaatgtctcaaagaaggaattcagaattctattagataaatttaacagacagattgaagtaattaaaaataatcaaacagaaattatggagcttaaaaacgcaattggcatactgaaaatgcatcagagttctttaatagcagcattgatcaaacagaagaaagaattagtgaacctgaagacagcctatttgaaaatacacagaggagacaaaagaaaaaaatataaaagaatgaagcacacctaa

SEQ ID NO: 16 - protein, 121 aa, Homo sapiens [sequence omitted]

SEQ ID NO: 17 - DNA, 348 bp, Homo sapiens
aacatgacatcaacaaaggaactaaataaggcaccagtaaacaatcctggagaaacagaactatgtgaccttttagacaaaaaattcaaaatagcagtgttgaggaaactaaaaggaattcaaaataacacagagaaggaattcagaattctatcagataaatttaacaaagagattgaaataattaaaaagaatcaagcagaaactctggagctaaaaaatgcagttggcacactaacaaaagcatcacagtcctttaaaagcagaatggatatagcagaaagaagaattagtgaacttaaagacaggctatttgaaaatacagtcagaagagaaaaaagaatataa

SEQ ID NO: 18 - protein, 114 aa, Homo sapiens [sequence omitted]

SEQ ID NO: 19 - DNA, 432 bp, Homo sapiens
aatatgacctcaccaaatgaactaaataaggcaccagggatcaatcctggggaaacagaaatatgtgacctttcagacagagaattcacaatagctgtttcgaggaagctaaacaaaatccaagataacatggagaaggaattcagaatcctatcagataaatttaatgaagagattgaaataattaaaaagaatcaagcagaaattctggagctgaaaaacgcaattgacatgttgaagaatgcatcagagaatctcaccagcagaactgatcaagcaagagaaataattagtaagcttgaagacaggctatttgaaaacacaaagtcagaggagacaaatggaaaaagaataaaatgcaatgaagcacacctacaagaactagaaaatagcttcaaaatgggaaatctaaaagttattggccttaaatag

SEQ ID NO: 20 - protein, 142 aa, Homo sapiens [sequence omitted]

SEQ ID NO: 21 - DNA, 252 bp, Homo sapiens
aacatgcccttaccaaatgagctaaataaggcgccagggaccaatcctggagaaacagagacatgtgacctttcagacagagaattcaaaatagctgtgttgagaaaactcaaagaaattcaagagaatacagacaaggaattgagaattctatcagataaatttaacaaagaaatcaaaataatgaaaaagaatcaagcagaaattctgaagctgaaaaattcaattagtataatgaagaatgcatcatag

SEQ ID NO: 22 - protein, 82 aa, Homo sapiens [sequence omitted]

SEQ ID NO: 23 - DNA, 327 bp, Homo sapiens
aacatgacctcaccaaatgaactgaataaggcaccagggacgaatttaggagaaacagagatttgtgacctttcagacagagaattcaagaaagctgtgttgcagaagctcaaagaaattcaagataacacagagaaggagttcagaattctattacataaatttaacaaagagattaaaataattaaaaagaatcaagcagaaattctagaagcaaaaaatgcaactgacatactgatgaatgcatcagaccctattaatagcacaattgatgaagcagaagaaagaattagtgagcttgaagacaggctatttgaaagtatatag

SEQ ID NO: 24 - protein, 107 aa, Homo sapiens [sequence omitted]

SEQ ID NO: 25 - DNA, 285 bp, Homo sapiens
aacatggcctcaccaaacaaactaaataaggcaccagaaaccaatcccaaagagacagaggtatgtgacctttcagacagagaactcaaaatacctgttttgaggaagttcaatgaaattcaagataacacagagaaggaattcagaattctatcagataaatttaacaaagagattgaaataattaaaaagaatcaagcggaaattccggaagtgaaaaatgcaattaatacactgaagaattcatcagagtctcttaatagcagaattgatcaagcagaataa

SEQ ID NO: 26 - protein, 93 aa, Homo sapiens [sequence omitted]

SEQ ID NO: 27 - DNA, 345 bp, Homo sapiens
aacatgacctcgcctaatgaactaaatgaagcaccagggaccaatcctgcagagacagagatatgtaacattttagacagagaattcaaaatagctgttttgaggaaactcaatgaaattcaagataacacagagaaggaattgaaggttctctcagataaaattatcaaagagattgaaataattaaaatgaatcaagcagaaattctggagttgaaaaatgcaactgacatacggaagaatgcatcggggtctcttaacaagagacttaatctttcagaagaaagaattagtgagctcggagatagcctatttgacaatatacagtcagaggaagcaaactaa

SEQ ID NO: 28 - protein, 113 aa, Homo sapiens [sequence omitted]

SEQ ID NO: 29 - DNA, 384 bp, Homo sapiens
aatatgacctcaccaaatgaactaaataaggtaccaggggccaatcctggagaaacagagatttgtgatcattcagaaagagaattcaaaataactgtcttgaggaaactcaaagacattcatgataacacagagaagacaatcagaattctatcagataaatttaacaaagatattgaaataattttaaaaaatcaagatgatattctggagctggaaaatgcaattggtgtactgaagaatgaatcagggttctttaatagcaggatggatgaagcagaagaaataattagaaagcttgaagacagtttatttgaaaatatacagtcagagaagaaagcgaaaaaagtaaaacaaacaaacaaaaaaagaagcatgtattag

SEQ ID NO: 30 - protein, 126 aa, Homo sapiens [sequence omitted]

SEQ ID NO: 31 - DNA, 237 bp, Homo sapiens
aacatgacctcaccaaataaacttaaaaaggcaccagggaccaatcctggagaaacagaaacatgtggactttcacagagagaattcaaagtagctgtgttgaggaaactcaaagaaattcaagataacagagagaaggaattcagaattgtatcagataaatttaacaaagagattgaaataattaaaaagaatcaggcagaaatactggagctgaaaaatcaactggcatactga

SEQ ID NO: 32 - protein, 77 aa, Homo sapiens [sequence omitted]

SEQ ID NO: 33 - DNA, 285 bp, Homo sapiens
aacatgacctcaccaaacaaactaaataaggcacccagggccaattctggagaaacagagatacgtaaactttcaaacacagaaatcaagatagctgtgttgagaaaactcaaagaaattcaagataacacagagaaagaattcagaattctatcagataaatttaacaaagagattgaaataactaaaaagaatcaagcagaaattctggagctgagaaatgcaattgacatactgaagaatgcatcagggtcttttaatagcagaattgagcaagcagaataa

SEQ ID NO: 34 - protein, 93 aa, Homo sapiens [sequence omitted]

SEQ ID NO: 35 - DNA, 285 bp, Homo sapiens
aacatgacctcactaaatgaactaaataaggcaccaggggccaaccctggagaaacagagatatgcgacctttcagacagagaattcaaaatagctgtgttggggaaattcaaagataacacagagaaggaattcagaattctatcagataaatttaacaaagagattgaaataattaaaaagaatcaagcagaaattctggagctgaaaaatgcaattgccacattaaagaatgcattagagttttttaatagcagaatttatggagcagaaaaaaagaattag

SEQ ID NO: 36 - protein, 93 aa, Homo sapiens [sequence omitted]

SEQ ID NO: 37 - DNA, 339 bp, Homo sapiens
tcaatggccagacacctacaaacatccactagcatcaagaccatccaggaaaataggacctcaccaagtgaactaaataaggcaccaggggccagtcttggagaaacagagatatgtgatctttcaaacagagaattgaaaatagctgttttgaggaaactcaaagaaattcaagatagcacagagaaggaattcagaatcctatcagataaatttaacaaacaaattgaaataattaaaaacagtcaagcagaaattctggagctgaaaaatgcaattgacttactgaagaatgcatcagaatctcctaatagtagaattaatcaagtagaagaatga

SEQ ID NO: 38 - protein, 111 aa, Homo sapiens
MARHLQTSTSIKTIQENRTSPSELNKAPGASLGETEICDLSNRELKIAVLRKLKEIQDSTEKEFRILSDKFNKQIEIIKNSQAEILELKNAIDLLKNASESPNSRINQVEE

SEQ ID NO: 39 - DNA, 345 bp, Homo sapiens
tcaatgccaagacaccaaagaacacctactagaatcaacaccatccaggaaaacacgacctcatcaaatgagctaaatgaggcaccagggatcactcctggagaaacagagatatgtgacctttcagacagagaattcaaagtagctgtgttgagagagctcaaagaaattcaagataacacagagaagaaattcagaattctaccagataaatttatcaaagagattgaaataattaaaaagaatcaatcagaaattctggagctgaaaaacccaactgctgtactgaagaatgcatcagagtcccttaatagcagaatggatcgagtagaaaagaagaattag

SEQ ID NO: 40 - protein, 113 aa, Homo sapiens [sequence omitted]

SEQ ID NO: 41 - DNA, 22 bp, Homo sapiens
aacatgacctcaccaaataaac

SEQ ID NO: 42 - DNA, 29 bp, Homo sapiens
tcatttttttttattccttttcttttgtc

SEQ ID NO: 43 - DNA, 21 bp, Homo sapiens
aacatgacatcaccaaatgag

SEQ ID NO: 44 - DNA, 22 bp, Homo sapiens
ttacaggtgcctgccactgcac

SEQ ID NO: 45 - DNA, 22 bp, Homo sapiens
aacatgacctcaccaaatgaac

SEQ ID NO: 46 - DNA, 24 bp, Homo sapiens
tcaagagactgatgcattctttag

SEQ ID NO: 47 - DNA, 22 bp, Homo sapiens
aacatgacctcaccaaatgaac

SEQ ID NO: 48 - DNA, 24 bp, Homo sapiens
ctaattccgattaattctactatg

SEQ ID NO: 49 - DNA, 21 bp, Homo sapiens
aacatgacctcaccaaatgag

SEQ ID NO: 50 - DNA, 25 bp, Homo sapiens
tcattgttttttgttgtttttggtc

SEQ ID NO: 51 - DNA, 22 bp, Homo sapiens
cacatgacctcaggaaatgaag

SEQ ID NO: 52 - DNA, 27 bp, Homo sapiens
ttattgttttttattctttttcttttg

SEQ ID NO: 53 - DNA, 22 bp, Homo sapiens
aacatgacctcaccaaatgaac

SEQ ID NO: 54 - DNA, 27 bp, Homo sapiens
tcaatcagttctgctattaaaaaactc

SEQ ID NO: 55 - DNA, 22 bp, Homo sapiens
aacatgacctcaccaaatgaac

SEQ ID NO: 56 - DNA, 30 bp, Homo sapiens
ttaggtgtgcttcattcttttatatttttt

SEQ ID NO: 57 - DNA, 22 bp, Homo sapiens
aacatgacatcaacaaaggaac

SEQ ID NO: 58 - DNA, 26 bp, Homo sapiens
ttatattcttttttctcttctgactg

SEQ ID NO: 59 - DNA, 22 bp, Homo sapiens
aatatgacctcaccaaatgaac

SEQ ID NO: 60 - DNA, 24 bp, Homo sapiens
ctatttaaggccaataacttttag

SEQ ID NO: 61 - DNA, 22 bp, Homo sapiens
aacatgcccttaccaaatgagc

SEQ ID NO: 62 - DNA, 23 bp, Homo sapiens
ctatgatgcattcttcattatac

SEQ ID NO: 63 - DNA, 22 bp, Homo sapiens
aacatgacctcaccaaatgaac

SEQ ID NO: 64 - DNA, 24 bp, Homo sapiens
ctatatactttcaaatagcctgtc

SEQ ID NO: 65 - DNA, 22 bp, Homo sapiens
aacatgacctcgcctaatgaac

SEQ ID NO: 66 - DNA, 21 bp, Homo sapiens
ttagtttgcttcctctgactg

SEQ ID NO: 67 - DNA, 22 bp, Homo sapiens
aatatgacctcaccaaatgaac

SEQ ID NO: 68 - DNA, 26 bp, Homo sapiens
ctaatacatgcttctttttttgtttg

SEQ ID NO: 69 - DNA, 22 bp, Homo sapiens
aacatgacctcaccaaataaac

SEQ ID NO: 70 - DNA, 25 bp, Homo sapiens
tcagtatgccagttgatttttcagc

SEQ ID NO: 71 - DNA, 22 bp, Homo sapiens
aacatgacctcaccaaacaaac

SEQ ID NO: 72 - DNA, 23 bp, Homo sapiens
ttattctgcttgctcaattctgc

SEQ ID NO: 73 - DNA, 22 bp, Homo sapiens
aacatgacctcactaaatgaac

SEQ ID NO: 74 - DNA, 29 bp, Homo sapiens
ctaattctttttttctgctccataaattc

SEQ ID NO: 75 - DNA, 23 bp, Homo sapiens
tcaatggccagacacctacaaac

SEQ ID NO: 76 - DNA, 26 bp, Homo sapiens
tcattcttctacttgattaattctac

SEQ ID NO: 77 - DNA, 23 bp, Homo sapiens
tcaatgccaagacaccaaagaac

SEQ ID NO: 78 - DNA, 25 bp, Homo sapiens
ctaattcttcttttctactcgatcc

SEQ ID NO: 79 - DNA, 39 bp, Artificial sequence
  ATT site-modified, ORF-specific CL primer
aagcaggcttcgccaccaacatgacctcaccaaataaac

SEQ ID NO: 80 - DNA, 41 bp, Artificial sequence
  ATT site-modified, ORF-specific CL primer
gtgatggtgatggtgtttttttttattccttttcttttgtc

SEQ ID NO: 81 - DNA, 38 bp, Artificial sequence
  ATT site-modified, ORF-specific CL primer
aagcaggcttcgccaccaacatgacatcaccaaatgag

SEQ ID NO: 82 - DNA, 34 bp, Artificial sequence
  ATT site-modified, ORF-specific CL primer
gtgatggtgatggtgcaggtgcctgccactgcac

SEQ ID NO: 83 - DNA, 36 bp, Artificial sequence
  ATT site-modified, ORF-specific CL primer
aagcaggcttcgccaccatgacctcaccaaatgaac

SEQ ID NO: 84 - DNA, 36 bp, Artificial sequence
  ATT site-modified, ORF-specific CL primer
gtgatggtgatggtgagagactgatgcattctttag

SEQ ID NO: 85 - DNA, 39 bp, Artificial sequence
  ATT site-modified, ORF-specific CL primer
aagcaggcttcgccaccaacatgacctcaccaaatgaac

SEQ ID NO: 86 - DNA, 36 bp, Artificial sequence
  ATT site-modified, ORF-specific CL primer
gtgatggtgatggtgattccgattaattctactatg

SEQ ID NO: 87 - DNA, 38 bp, Artificial sequence
  ATT site-modified, ORF-specific CL primer
aagcaggcttcgccaccaacatgacctcaccaaatgag

SEQ ID NO: 88 - DNA, 37 bp, Artificial sequence
  ATT site-modified, ORF-specific CL primer
gtgatggtgatggtgttgttttttgttgtttttggtc

SEQ ID NO: 89 - DNA, 39 bp, Artificial sequence
  ATT site-modified, ORF-specific CL primer
aagcaggcttcgccacccacatgacctcaggaaatgaag

SEQ ID NO: 90 - DNA, 39 bp, Artificial sequence
  ATT site-modified, ORF-specific CL primer
gtgatggtgatggtgttgttttttattctttttcttttg

SEQ ID NO: 91 - DNA, 39 bp, Artificial sequence; identical to SEQ ID NO: 85
  ATT site-modified, ORF-specific CL primer

SEQ ID NO: 92 - DNA, 39 bp, Artificial sequence
  ATT site-modified, ORF-specific CL primer
gtgatggtgatggtgatcagttctgctattaaaaaactc

SEQ ID NO: 93 - DNA, 39 bp, Artificial sequence; identical to SEQ ID NO: 85
  ATT site-modified, ORF-specific CL primer

SEQ ID NO: 94 - DNA, 42 bp, Artificial sequence
  ATT site-modified, ORF-specific CL primer
gtgatggtgatggtgggtgtgcttcattcttttatatttttt

SEQ ID NO: 95 - DNA, 39 bp, Artificial sequence
  ATT site-modified, ORF-specific CL primer
aagcaggcttcgccaccaacatgacatcaacaaaggaac

SEQ ID NO: 96 - DNA, 38 bp, Artificial sequence
  ATT site-modified, ORF-specific CL primer
gtgatggtgatggtgtattcttttttctcttctgactg

SEQ ID NO: 97 - DNA, 39 bp, Artificial sequence
  ATT site-modified, ORF-specific CL primer
aagcaggcttcgccaccaatatgacctcaccaaatgaac

SEQ ID NO: 98 - DNA, 36 bp, Artificial sequence
  ATT site-modified, ORF-specific CL primer
gtgatggtgatggtgtttaaggccaataacttttag

SEQ ID NO: 99 - DNA, 39 bp, Artificial sequence
  ATT site-modified, ORF-specific CL primer
aagcaggcttcgccaccaacatgcccttaccaaatgagc

SEQ ID NO: 100 - DNA, 35 bp, Artificial sequence
  ATT site-modified, ORF-specific CL primer
gtgatggtgatggtgtgatgcattcttcattatac

SEQ ID NO: 101 - DNA, 39 bp, Artificial sequence; identical to SEQ ID NO: 85
  ATT site-modified, ORF-specific CL primer

SEQ ID NO: 102 - DNA, 36 bp, Artificial sequence
  ATT site-modified, ORF-specific CL primer
gtgatggtgatggtgtatactttcaaatagcctgtc

SEQ ID NO: 103 - DNA, 39 bp, Artificial sequence
  ATT site-modified, ORF-specific CL primer
aagcaggcttcgccaccaacatgacctcgcctaatgaac

SEQ ID NO: 104 - DNA, 33 bp, Artificial sequence
  ATT site-modified, ORF-specific CL primer
gtgatggtgatggtggtttgcttcctctgactg

SEQ ID NO: 105 - DNA, 39 bp, Artificial sequence; identical to SEQ ID NO: 97
  ATT site-modified, ORF-specific CL primer

SEQ ID NO: 106 - DNA, 38 bp, Artificial sequence
  ATT site-modified, ORF-specific CL primer
gtgatggtgatggtgatacatgcttctttttttgtttg

SEQ ID NO: 107 - DNA, 39 bp, Artificial sequence; identical to SEQ ID NO: 79
  ATT site-modified, ORF-specific CL primer

SEQ ID NO: 108 - DNA, 37 bp, Artificial sequence
  ATT site-modified, ORF-specific CL primer
gtgatggtgatggtggtatgccagttgatttttcagc

SEQ ID NO: 109 - DNA, 39 bp, Artificial sequence
  ATT site-modified, ORF-specific CL primer
aagcaggcttcgccaccaacatgacctcaccaaacaaac

SEQ ID NO: 110 - DNA, 35 bp, Artificial sequence
  ATT site-modified, ORF-specific CL primer
gtgatggtgatggtgttctgcttgctcaattctgc

SEQ ID NO: 111 - DNA, 39 bp, Artificial sequence
  ATT site-modified, ORF-specific CL primer
aagcaggcttcgccaccaacatgacctcactaaatgaac

SEQ ID NO: 112 - DNA, 41 bp, Artificial sequence
  ATT site-modified, ORF-specific CL primer
gtgatggtgatggtgattctttttttctgctccataaattc

SEQ ID NO: 113 - DNA, 40 bp, Artificial sequence
  ATT site-modified, ORF-specific CL primer
aagcaggcttcgccacctcaatggccagacacctacaaac

SEQ ID NO: 114 - DNA, 38 bp, Artificial sequence
  ATT site-modified, ORF-specific CL primer
gtgatggtgatggtgttcttctacttgattaattctac

SEQ ID NO: 115 - DNA, 40 bp, Artificial sequence
  ATT site-modified, ORF-specific CL primer
aagcaggcttcgccacctcaatgccaagacaccaaagaac

SEQ ID NO: 116 - DNA, 37 bp, Artificial sequence
  ATT site-modified, ORF-specific CL primer
gtgatggtgatggtgattcttcttttctactcgatcc

SEQ ID NO: 117 - DNA, 59 bp, Artificial sequence
  His tag-modified, ORF-specific CL primer
ggggacaagtttgtacaaaaaagcaggcttcgccaccaacatgacctcaccaaataaac

SEQ ID NO: 118 - DNA, 77 bp, Artificial sequence
  His tag-modified, ORF-specific CL primer
ggggaccactttgtacaagaaagctgggtttcaatggtgatggtgatggtgtttttttttattccttttcttttgtc

SEQ ID NO: 119 - DNA, 58 bp, Artificial sequence
  His tag-modified, ORF-specific CL primer
ggggacaagtttgtacaaaaaagcaggcttcgccaccaacatgacatcaccaaatgag

SEQ ID NO: 120 - DNA, 70 bp, Artificial sequence
  His tag-modified, ORF-specific CL primer
ggggaccactttgtacaagaaagctgggtttcaatggtgatggtgatggtgcaggtgcctgccactgcac

SEQ ID NO: 121 - DNA, 59 bp, Artificial sequence
  His tag-modified, ORF-specific CL primer
ggggacaagtttgtacaaaaaagcaggcttcgccaccaacatgacctcaccaaatgaac

SEQ ID NO: 122 - DNA, 72 bp, Artificial sequence
  His tag-modified, ORF-specific CL primer
ggggaccactttgtacaagaaagctgggtttcaatggtgatggtgatggtgagagactgatgcattctttag

SEQ ID NO: 123 - DNA, 59 bp, Artificial sequence; identical to SEQ ID NO: 121
  His tag-modified, ORF-specific CL primer

SEQ ID NO: 124 - DNA, 72 bp, Artificial sequence
  His tag-modified, ORF-specific CL primer
ggggaccactttgtacaagaaagctgggtttcaatggtgatggtgatggtgattccgattaattctactatg

SEQ ID NO: 125 - DNA, 58 bp, Artificial sequence
  His tag-modified, ORF-specific CL primer
ggggacaagtttgtacaaaaaagcaggcttcgccaccaacatgacctcaccaaatgag

SEQ ID NO: 126 - DNA, 73 bp, Artificial sequence
  His tag-modified, ORF-specific CL primer
ggggaccactttgtacaagaaagctgggtttcaatggtgatggtgatggtgttgttttttgttgtttttggtc

SEQ ID NO: 127 - DNA, 59 bp, Artificial sequence
  His tag-modified, ORF-specific CL primer
ggggacaagtttgtacaaaaaagcaggcttcgccacccacatgacctcaggaaatgaag

SEQ ID NO: 128 - DNA, 75 bp, Artificial sequence
  His tag-modified, ORF-specific CL primer
ggggaccactttgtacaagaaagctgggtttcaatggtgatggtgatggtgttgttttttattctttttcttttg

SEQ ID NO: 129 - DNA, 59 bp, Artificial sequence; identical to SEQ ID NO: 121
  His tag-modified, ORF-specific CL primer

SEQ ID NO: 130 - DNA, 75 bp, Artificial sequence
  His tag-modified, ORF-specific CL primer
ggggaccactttgtacaagaaagctgggtttcaatggtgatggtgatggtgatcagttctgctattaaaaaactc

SEQ ID NO: 131 - DNA, 59 bp, Artificial sequence; identical to SEQ ID NO: 121
  His tag-modified, ORF-specific CL primer

SEQ ID NO: 132 - DNA, 78 bp, Artificial sequence
  His tag-modified, ORF-specific CL primer
ggggaccactttgtacaagaaagctgggtttcaatggtgatggtgatggtgggtgtgcttcattcttttatatttttt

SEQ ID NO: 133 - DNA, 59 bp, Artificial sequence
  His tag-modified, ORF-specific CL primer
ggggacaagtttgtacaaaaaagcaggcttcgccaccaacatgacatcaacaaaggaac

SEQ ID NO: 134 - DNA, 74 bp, Artificial sequence
  His tag-modified, ORF-specific CL primer
ggggaccactttgtacaagaaagctgggtttcaatggtgatggtgatggtgtattcttttttctcttctgactg

SEQ ID NO: 135 - DNA, 59 bp, Artificial sequence
  His tag-modified, ORF-specific CL primer
ggggacaagtttgtacaaaaaagcaggcttcgccaccaatatgacctcaccaaatgaac

SEQ ID NO: 136 - DNA, 72 bp, Artificial sequence
  His tag-modified, ORF-specific CL primer
ggggaccactttgtacaagaaagctgggtttcaatggtgatggtgatggtgtttaaggccaataacttttag

SEQ ID NO: 137 - DNA, 59 bp, Artificial sequence
  His tag-modified, ORF-specific CL primer
ggggacaagtttgtacaaaaaagcaggcttcgccaccaacatgcccttaccaaatgagc

SEQ ID NO: 138 - DNA, 71 bp, Artificial sequence
  His tag-modified, ORF-specific CL primer
ggggaccactttgtacaagaaagctgggtttcaatggtgatggtgatggtgtgatgcattcttcattatac

SEQ ID NO: 139 - DNA, 59 bp, Artificial sequence; identical to SEQ ID NO: 121
  His tag-modified, ORF-specific CL primer

SEQ ID NO: 140 - DNA, 72 bp, Artificial sequence
  His tag-modified, ORF-specific CL primer
ggggaccactttgtacaagaaagctgggtttcaatggtgatggtgatggtgtatactttcaaatagcctgtc

SEQ ID NO: 141 - DNA, 59 bp, Artificial sequence
  His tag-modified, ORF-specific CL primer
ggggacaagtttgtacaaaaaagcaggcttcgccaccaacatgacctcgcctaatgaac

SEQ ID NO: 142 - DNA, 69 bp, Artificial sequence
  His tag-modified, ORF-specific CL primer
ggggaccactttgtacaagaaagctgggtttcaatggtgatggtgatggtggtttgcttcctctgactg

SEQ ID NO: 143 - DNA, 59 bp, Artificial sequence; identical to SEQ ID NO: 135
  His tag-modified, ORF-specific CL primer

SEQ ID NO: 144 - DNA, 74 bp, Artificial sequence
  His tag-modified, ORF-specific CL primer
ggggaccactttgtacaagaaagctgggtttcaatggtgatggtgatggtgatacatgcttctttttttgtttg

SEQ ID NO: 145 - DNA, 59 bp, Artificial sequence; identical to SEQ ID NO: 117
  His tag-modified, ORF-specific CL primer

SEQ ID NO: 146 - DNA, 73 bp, Artificial sequence
  His tag-modified, ORF-specific CL primer
ggggaccactttgtacaagaaagctgggtttcaatggtgatggtgatggtggtatgccagttgatttttcagc

SEQ ID NO: 147 - DNA, 59 bp, Artificial sequence
  His tag-modified, ORF-specific CL primer
ggggacaagtttgtacaaaaaagcaggcttcgccaccaacatgacctcaccaaacaaac

SEQ ID NO: 148 - DNA, 71 bp, Artificial sequence
  His tag-modified, ORF-specific CL primer
ggggaccactttgtacaagaaagctgggtttcaatggtgatggtgatggtgttctgcttgctcaattctgc

SEQ ID NO: 149 - DNA, 59 bp, Artificial sequence
  His tag-modified, ORF-specific CL primer
ggggacaagtttgtacaaaaaagcaggcttcgccaccaacatgacctcactaaatgaac

SEQ ID NO: 150 - DNA, 77 bp, Artificial sequence
  His tag-modified, ORF-specific CL primer
ggggaccactttgtacaagaaagctgggtttcaatggtgatggtgatggtgattctttttttctgctccataaattc

SEQ ID NO: 151 - DNA, 60 bp, Artificial sequence
  His tag-modified, ORF-specific CL primer
ggggacaagtttgtacaaaaaagcaggcttcgccacctcaatggccagacacctacaaac

SEQ ID NO: 152 - DNA, 74 bp, Artificial sequence
  His tag-modified, ORF-specific CL primer
ggggaccactttgtacaagaaagctgggtttcaatggtgatggtgatggtgttcttctacttgattaattctac

SEQ ID NO: 153 - DNA, 60 bp, Artificial sequence
  His tag-modified, ORF-specific CL primer
ggggacaagtttgtacaaaaaagcaggcttcgccacctcaatgccaagacaccaaagaac

SEQ ID NO: 154 - DNA, 73 bp, Artificial sequence
  His tag-modified, ORF-specific CL primer
ggggaccactttgtacaagaaagctgggtttcaatggtgatggtgatggtgattcttcttttctactcgatcc

SEQ ID NO: 155 - protein, 78 aa, Homo sapiens [sequence omitted]

SEQ ID NO: 156 - protein, 75 aa, Artificial sequence
  Human pIFNFH polypeptides consensus sequence
  misc_feature: pIFNFH consensus sequence
MTSPNELNKAPGTNPGETEICDLSDREFRIAVLRKLKEIQDNTEKEFRILSDKFNKEIEIIKKNQAEILELKNAI

SEQ ID NO: 157 - protein, 154 aa, Homo sapiens
MGLRCDSETSWLQVRFSTIPAVVATGTDFFCPRKVEEKVKRTLSCTSGTSSATESIKWALGAPDSRTWLLDGISGPALGQRGAHCPQRHRQTSTSIKTIQENMTSSNKLNKAPGTNPGETEICDFSDREIKMAVLRKVKEIQDNTEKEFRILSD

SEQ ID NO: 158 - protein, 71 aa, Homo sapiens
GEFKIAVLRKLKEIQDNKEKDFRILSDKFNEEIEIIKKNQSEIQGLKNAIHILTNASESFNSRIDQAEEII